(19) United States
(12) Patent Application Publication
(10) Pub. No.: US 2026/0294460 A1
(43) Pub. Date: Oct. 1, 2026

(54) SYSTEMS AND METHODS OF HUMERAL HEAD SURGICAL GUIDE AND APPARATUSES

(71) Applicant: Glenoid Solutions, LLC, Tampa, FL (US)

(72) Inventors: Sergio Gutiérrez, Tampa, FL (US); Jonathan Chad Levy, Fort Lauderdale, FL (US)

(73) Assignee: Glenoid Solutions, LLC, Tampa, FL (US)

(21) Appl. No.: 19/632,006

(22) Filed: Mar. 27, 2026

Related U.S. Application Data
(60) Provisional application No. 63/780,964, filed on Mar. 31, 2025.

(51) Int. Cl. A61B 17/32; A61B 17/00; A61B 90/00

(52) U.S. Cl. CPC A61B 17/32; A61B 90/08; A61B 2017/00477; A61B 2017/320052; A61B 2090/0807

(57) ABSTRACT

A humeral head surgical guide can include a guide body including a first marker and a second marker and defining a guide aperture extending along a first axis at a center of the guide body. The humeral head surgical guide can include a first movable guide member coupled to the guide body comprising one or more first indicators and a first supporting member including at least one first engagement feature. The humeral head surgical guide can include a second movable guide member coupled to the guide body including one or more second indicators and a second supporting member including at least one second engagement feature. The first marker and the second marker can be oriented relative to the one or more first or second indicators to indicate a first or second distance between the first axis and the at least one first or second engagement feature, respectively.

### CROSS-REFERENCE TO RELATED APPLICATION

The present application claims priority to U.S. Provisional Patent Application No. 63/780,964, filed on Mar. 31, 2025, the entire disclosure of which is incorporated herein by reference for all purposes.

### TECHNICAL FIELD

The present disclosure relates generally to systems and methods of humeral head surgical guide and apparatuses.

### BACKGROUND

Shoulder arthroplasty is a surgical procedure that involves replacing damaged portions of the shoulder joint with prosthetic implants. However, preparing the joint and selecting an implant may be difficult due to variations in anatomy.

### SUMMARY

At least one aspect relates to a humeral head surgical guide. The humeral head surgical guide can include a guide body including a first marker and a second marker. The guide body can define a guide aperture extending along a first axis at a center of the guide body. The humeral head surgical guide can include a first movable guide member coupled to the guide body including one or more first indicators. The first movable guide member can move along a second axis perpendicular to the first axis. The humeral head surgical guide can include a first supporting member including at least one first engagement feature. The first supporting member can be coupled to and extend from the first movable guide member. The first marker can be oriented relative to the one or more first indicators to indicate a first distance between the first axis and the at least one first engagement feature. The humeral head surgical guide can include a second movable guide member coupled to the guide body including one or more second indicators. The second movable guide member can move along a third axis perpendicular to the first axis and relative to the second axis. The humeral head surgical guide can include a second supporting member including at least one second engagement feature. The second supporting member can be coupled to and extend from the second movable guide member. The second marker can be oriented relative to the one or more second indicators to indicate a second distance between the first axis and the at least one second engagement feature.

In some implementations, the guide body includes a third marker. In some implementations, the humeral head surgical guide can include a third movable guide member coupled to the guide body including one or more third indicators. In some implementations, the third movable guide member can move along the second axis. In some implementations, the humeral head surgical guide can include a third supporting member including at least one third engagement feature. In some implementations, the third supporting member can be coupled to and extend from the third movable guide member. In some implementations, the third marker can be oriented relative to the one or more third indicators to indicate a third distance between the first axis and the at least one third engagement feature.

In some implementations, the guide body includes a fourth marker. In some implementations, the humeral head surgical guide can include a fourth movable guide member coupled to the guide body including one or more fourth indicators. In some implementations, the fourth movable guide member can move along the third axis. In some implementations, the humeral head surgical guide can include a fourth supporting member including at least one fourth engagement feature. In some implementations, the fourth supporting member can be coupled to and extend from the fourth movable guide member. In some implementations, the fourth marker can be oriented relative to the one or more fourth indicators to indicate a fourth distance between the first axis and the at least one fourth engagement feature.

In some implementations, the guide body is configured to receive a guide wire extending through the guide aperture. In some implementations, the guide wire is attachable to a center of a humeral head using at least the first movable guide member and the second movable guide member.

In some implementations, the humeral head surgical guide can include a shaft defining a shaft aperture. In some implementations, the guide wire can extend through the shaft aperture. In some implementations, the shaft can comprise one or more fifth indicators, a first end, and a second end opposite the first end. In some implementations, the one or more fifth indicators can indicate a fifth distance between the second end and at least one of the at least one first engagement feature or the at least one second engagement feature. In some implementations, the fifth distance indicates a depth of a humeral head.

In some implementations, the first movable guide member and the second movable guide member are independently movable.

In some implementations, the humeral head surgical guide can include a gear coupled with the first movable guide member and the second movable guide member. In some implementations, the gear can maintain the at least one first engagement feature and the at least one second engagement feature at a same distance from the first axis. In some implementations, the gear can cause the first movable guide member and the second movable guide member to move at a same rate.

In some implementations, the at least one first engagement feature and the at least one second engagement feature engage with different portions of a bottom surface of a humeral head.

In some implementations, the humeral head surgical guide can include a first extender coupled to and extending from the first movable guide member. In some implementations, the first marker is indicative of a first anatomical location on a humeral head. In some implementations, the humeral head surgical guide can include a second extender coupled to and extending from the second movable guide member. In some implementations, the second marker is indicative of a second anatomical location on the humeral head.

In some implementations, the humeral head surgical guide further includes a base marker coupled to and located on the guide body, a first reference marker coupled to and located on the first extender, where the base marker and the first reference marker are scanned using at least one sensor to determine at least the first distance, a second reference marker coupled to and located on the second extender, where the base marker and the second reference marker are scanned using the at least one sensor to determine at least a fourth distance, a shaft coupled to the guide body, a third handle coupled to and extending around the shaft, and a third reference marker coupled to and located on the third handle, where the base marker and the third reference marker are scanned using the at least one sensor to determine at least a fifth distance.

At least one aspect relates to a humeral head cutting apparatus. The humeral head cutting apparatus can include a first body defining a first aperture. The humeral head cutting apparatus can include a first movable member extending around the first body. The first movable member can be movable along the first body. The humeral head cutting apparatus can include a second body coupled to the first movable member. The humeral head cutting apparatus can include a second movable member extending around the second body. The second movable member can be movable along the second body. The humeral head cutting apparatus can include an arm coupled to the second movable member. The humeral head cutting apparatus can include a cutting support member removably coupled with the arm. The cutting support member can define a plurality of cutting support apertures. The cutting support member can include a top surface that guides a cutting member. The cutting support member can include a plurality of pins extending through a subset of the plurality of cutting support apertures. The plurality of pins can secure the cutting support member to a humeral head.

In some implementations, the plurality of cutting support apertures are defined at different positions on the cutting support member.

In some implementations, the humeral head cutting apparatus can include a first fastener coupled to the first movable member. In some implementations, the humeral head cutting apparatus can include a second fastener coupled to the second movable member. In some implementations, the humeral head cutting apparatus can include a third fastener coupled to the cutting supporting member.

In some implementations, the humeral head cutting apparatus can include a platform removably coupled with the cutting support member and facing the top surface. In some implementations, the cutting member can extend between the top surface and the platform. In some implementations, the humeral head cutting apparatus can include a fourth fastener coupled to the platform. In some implementations, the fourth fastener can removably couple the platform from the cutting support member.

In some implementations, the first aperture receives a guide wire attached to a center of the humeral head.

In some implementations, the body includes one or more first indicators indicating a depth. In some implementations, a position of the first movable member along the first body is set to one of the one or more first indicators corresponding to a depth of the humeral head.

In some implementations, the humeral head cutting apparatus further includes a base marker coupled to and located on the first body and a reference marker associated with an angle of the cutting support member relative to the first body, where the base marker and the reference marker are scanned to determine the angle.

At least one aspect relates to a humeral head coring apparatus. The humeral head coring apparatus can include a body including a first portion and a second portion contiguous with the first portion. The first portion can define a track. The body can define a body aperture extending along a first axis through the first portion and the second portion. The body aperture can receive a guide wire attached to a center of a humeral head. The humeral head coring apparatus can include a coring member coupled to the second portion. The coring member can comprise a plurality of cutting features. The coring member can core at least a portion of the humeral head using the plurality of cutting features. The humeral head coring apparatus can include an adjuster coupled to and movable along the track. The humeral head coring apparatus can include a receiving member coupled to the adjuster. The receiving member can define a cutting depth of the coring member. The receiving member can be movable by the adjuster to define the cutting depth.

In some implementations, the first portion includes one or more indicators along the track. In some implementations, the one or more indicators correspond to the cutting depth.

In some implementations, the second portion receives the portion of the humeral head. In some implementations, the portion has a height corresponding to the cutting depth. In some implementations, the receiving member contacts the portion.

In some implementations, a diameter of the second portion is greater than a diameter of the first portion.

At least one aspect relates to a surgical kit. The surgical kit can include a guide wire. The surgical kit can include a humeral head surgical guide. The humeral head surgical guide can include a guide body including a first marker and a second marker. The guide body can define a guide aperture extending along a first axis at a center of the guide body. The guide aperture can receive the guide wire. The humeral head surgical guide can include a first movable guide member coupled to the guide body including one or more first indicators. The first movable guide member can move along a second axis perpendicular to the first axis. The humeral head surgical guide can include a first supporting member including at least one first engagement feature. The first supporting member can be coupled to and extend from the first movable guide member. The first marker can be oriented relative to the one or more first indicators to indicate a first distance between the first axis and the at least one first engagement feature. The humeral head surgical guide can include a second movable guide member coupled to the guide body including one or more second indicators. The second movable guide member can move along a third axis perpendicular to the first axis and relative to the second axis. The humeral head surgical guide can include a second supporting member including at least one second engagement feature. The second supporting member can be coupled to and extend from the second movable guide member. The second marker can be oriented relative to the one or more second indicators to indicate a second distance between the first axis and the at least one second engagement feature. The surgical kit can include a first surgical apparatus. The first surgical apparatus can include a first body defining a first aperture. The guide wire can extend through the first aperture. The first surgical apparatus can include a first movable member extending around the first body. The first movable member can be movable along the first body. The first surgical apparatus can include a cutting support member removably coupled with the first movable member. The cutting support member can guide a cutting member in cutting the humeral head. The surgical kit can include a second surgical apparatus. The second surgical apparatus can include a body including a first portion and a second portion contiguous with the first portion. The first portion can define a track. The body can define a body aperture extending along a first axis through the first portion and the second portion. The body aperture can receive a guide wire attached to a center of a humeral head. The second surgical apparatus can include a coring member coupled to the second portion. The coring member can comprise a plurality of cutting features. The coring member can core at least a portion of the humeral head using the plurality of cutting features.

In some implementations, the first surgical apparatus can include a second body coupled to the first movable member. In some implementations, the first surgical apparatus can include a second movable member extending around the second body. In some implementations, the second movable member can be movable along the second body. In some implementations, the first surgical apparatus can include an arm coupled to the second movable member. In some implementations, the cutting support member is removably coupled to the arm.

In some implementations, the cutting support member defines a plurality of cutting support apertures. In some implementations, the cutting support member can include a top surface that guides the cutting member. In some implementations, the cutting support member can include a plurality of pins extending through a subset of the plurality of cutting support apertures. In some implementations, the plurality of pins can secure the cutting support member to the humeral head.

In some implementations, the second surgical apparatus can include an adjuster coupled to the track. In some implementations, the adjuster can be movable along the track. In some implementations, the second surgical apparatus can include a receiving member coupled to the adjuster. In some implementations, the receiving member can define a cutting depth of the coring member. In some implementations, the receiving member can be movable by the adjuster to define the cutting depth.

In some implementations, the first distance and the second distance indicate one or more dimensions of the humeral head. In some implementations, the surgical kit can include an implant. In some implementations, the implant has dimensions corresponding to the one or more dimensions.

In some implementations the humeral head surgical guide further includes a first extender coupled to and extending from the first movable guide member, the first extender indicative of a first anatomical location on a humeral head and a second extender coupled to and extending from the second movable guide member, the second extender indicative of a second anatomical location on the humeral head.

In some implementations, the humeral head surgical guide further includes a base marker coupled to and located on the guide body, a first reference marker coupled to and located on the first extender, where the base marker and the first reference marker are scanned using at least one sensor to determine at least the first distance, a second reference marker coupled to and located on the second extender, where the base marker and the second reference marker are scanned using the at least one sensor to determine at least a fourth distance, a shaft coupled to the guide body, a third handle coupled to and extending around the shaft, and a third reference marker coupled to and located on the third handle, where the base marker and the third reference marker are scanned using the at least one sensor to determine at least a fifth distance.

In some implementations, the first surgical apparatus further includes a base marker coupled to and located on the first body and a reference marker associated with an angle of the cutting support member relative to the first body, where the base marker and the reference marker are scanned to determine the angle.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE DRAWINGS

The foregoing and other features of the present disclosure will become more fully apparent from the following description and appended claims taken in conjunction with the accompanying drawings. Understanding that these drawings depict only several implementations in accordance with the disclosure and are therefore not to be considered limiting of its scope, the disclosure will be described with additional specificity and detail through use of the accompanying drawings.

FIG. 1 is an illustration of a shoulder joint showing relevant anatomical features, in accordance with some implementations of the present disclosure.

FIG. 2 is a top view of a humeral head centering jig with tracking markers and humeral head retractor positioned on a humeral head, in accordance with some implementations of the present disclosure.

FIG. 3 is a detailed top view of the humeral head centering jig, in accordance with some implementations of the present disclosure.

FIG. 4 is a perspective view of the humeral head centering jig, in accordance with some implementations of the present disclosure.

FIG. 5 is another perspective view of the humeral head centering jig, in accordance with some implementations of the present disclosure.

FIG. 6 is an exploded view of the humeral head centering jig showing individual components, in accordance with some implementations of the present disclosure.

FIG. 7 is a perspective view of an alternative embodiment of the humeral head centering jig, in accordance with some implementations of the present disclosure.

FIG. 8 is an exploded view of the alternative embodiment of the humeral head centering jig, in accordance with some implementations of the present disclosure.

FIG. 9 is a perspective view showing the humeral head centering jig with a manipulating handle and humeral head retractor positioned on a humeral head, in accordance with some implementations of the present disclosure.

FIG. 10 is a perspective view showing a fixed humeral head cutting jig positioned on a humeral head, in accordance with some implementations of the present disclosure.

FIG. 11 is a perspective view showing an adjustable humeral head cutting jig positioned on a humeral head, in accordance with some implementations of the present disclosure.

FIG. 12 is a perspective view showing a humeral head coring drill positioned on a humeral head, in accordance with some implementations of the present disclosure.

FIG. 13 is an exploded view of the humeral head coring drill, in accordance with some implementations of the present disclosure.

FIG. 14 is a perspective view of an example humeral head surgical guide, in accordance with some implementations of the present disclosure.

FIG. 15 is a perspective view of the humeral head surgical guide of FIG. 14, in accordance with some implementations of the present disclosure.

FIG. 16 is a close-up view of the humeral head surgical guide of FIG. 15, in accordance with some implementations of the present disclosure.

FIG. 17 is a cross-sectional view of the humeral head surgical guide of FIG. 15, in accordance with some implementations of the present disclosure.

FIG. 18 is a perspective view of an example engagement feature, in accordance with some implementations of the present disclosure.

FIG. 19 is a perspective view of another example engagement feature, in accordance with some implementations of the present disclosure.

FIG. 20 is a perspective view of an example humeral head surgical guide, in accordance with some implementations of the present disclosure.

FIG. 21 is a flow diagram of an example method for using an example humeral head surgical guide, in accordance with some implementations of the present disclosure.

FIG. 22 is a perspective view of an example humeral head cutting apparatus, in accordance with some implementations of the present disclosure.

FIG. 23 is another perspective view of the humeral head cutting apparatus of FIG. 22, in accordance with some implementations of the present disclosure.

FIG. 24 is a cross-sectional view of the humeral head cutting apparatus of FIG. 22, in accordance with some implementations of the present disclosure.

FIG. 25 is a perspective view of a portion of the humeral head cutting apparatus of FIG. 22 guiding a cutting member, in accordance with some implementations of the present disclosure.

FIG. 26 is a perspective view of another example humeral head cutting apparatus, in accordance with some implementations of the present disclosure.

FIG. 27 is a perspective view of another example humeral head cutting apparatus, in accordance with some implementations of the present disclosure.

FIG. 28 is a perspective view of another example humeral head cutting apparatus, in accordance with some implementations of the present disclosure.

FIG. 29 is a perspective view of an example humeral head coring apparatus, in accordance with some implementations of the present disclosure.

FIG. 30 is a cross-sectional view of the humeral head coring apparatus of FIG. 29, in accordance with some implementations of the present disclosure.

FIG. 31 is a flow diagram of an example method for humeral head preparation, in accordance with some implementations of the present disclosure.

### DETAILED DESCRIPTION

Following below are more detailed descriptions of various concepts related to, and implementations of a humeral head surgical guide and apparatuses. The various concepts introduced above and discussed in greater detail below can be implemented in any of a number of ways, as the described concepts are not limited to any particular manner of implementation. Examples of specific implementations and applications are provided primarily for illustrative purposes.

Shoulder arthroplasty is commonly performed to address conditions such as severe arthritis, irreparable rotator cuff tears, or traumatic injuries. Implant positioning and implantation includes preparation of the humeral bone for long-term clinical outcomes (e.g., minimal complications following procedure, etc.). The preparation can include centering and establishing a reference axis perpendicular to an anatomical neck of the humerus, measurement of the humeral head dimensions, resection of the humeral head, and preparation of the humeral metaphysis and/or canal. Approaches to humeral preparation involve separate instruments for each of these steps, forcing the surgeon to repeatedly remove, reposition (e.g., recenter, etc.) reattach different devices during the procedure. For example, some approaches lack centering devices and may force surgeons to manually estimate placement of a cutting guide, introducing error and accuracy in cutting and resecting the humeral head.

In addition, establishing accurate centering on the humeral head presents several challenges. Many centering guides rely on limited anatomical landmarks and fail to accommodate patient-specific anatomical variations. These limited anatomical landmarks may be distorted by arthritis, trauma, osteophytes, or prior surgeries, and thus may not reliably represent a true center of rotation or reference point for patients with anatomical variations. In addition, anatomy can vary from patient to patient, including but not limited to humeral head size, offset, and inclination, and thus guides based on fixed reference points may lead to inaccurate positioning of the implant and increased risk of instability or early wear of the implant. Additionally, some instruments often do not provide accurate measurements of humeral head dimensions in multiple planes, which are used for selecting appropriately sized implants. Without such measurements, the likelihood of implant mismatch, suboptimal joint congruency, and compromised range of motion and function increases.

Humeral cutting apparatuses present additional challenges. Such devices often provide limited adjustability for controlling inclination angles, and thus surgeons may be limited to a set range of angles which may not accommodate the anatomy of the patient or a desired cutting angle plane. Furthermore, many cutting guides are unstable during the cutting process, potentially resulting in inaccurate resections. The ability to create precisely angled cuts for autograft harvesting in cases of glenoid bone loss is particularly limited with such instruments. For humeral preparation and autograft harvesting, reamers and drills often lack precise depth control and may not maintain proper alignment with the previously established reference axis. The ability to extract a properly sized bone core for autografting while preserving the structural integrity of the surrounding bone is particularly challenging with such instruments.

The systems and methods of the present disclosure address the aforementioned challenges by providing a kit of interconnected surgical guides and apparatuses for humeral preparation during shoulder arthroplasty. The systems and methods include a humeral head surgical guide which can determine and establish a center of the humeral head, allowing for drilling of a guide wire into the center of the humeral head for humeral preparation. In addition, the surgical guide can determine dimensions of the humeral head, allowing for accurate selection of an appropriate implant. Subsequent cutting and coring apparatus can be connected to the guide wire to establish a reference point for preparing the humerus for the implant, therefore removing repositioning and ensuring that cutting and coring to prepare the humerus uses the center of the humeral head as a reference point.

The systems and methods of the present disclosure improve accuracy in establishing the central axis of the humeral head, enhance measurement capabilities for implant sizing, provide precise control of resection thickness and angle, allow for the ability to harvest appropriately sized autografts for glenoid reconstruction, and provide consistent alignment between preparation steps. Such advantages allow for more accurate implant positioning even with variations in patient anatomy and improved options for addressing complex cases involving glenoid bone loss, potentially improving patient outcomes and implant longevity.

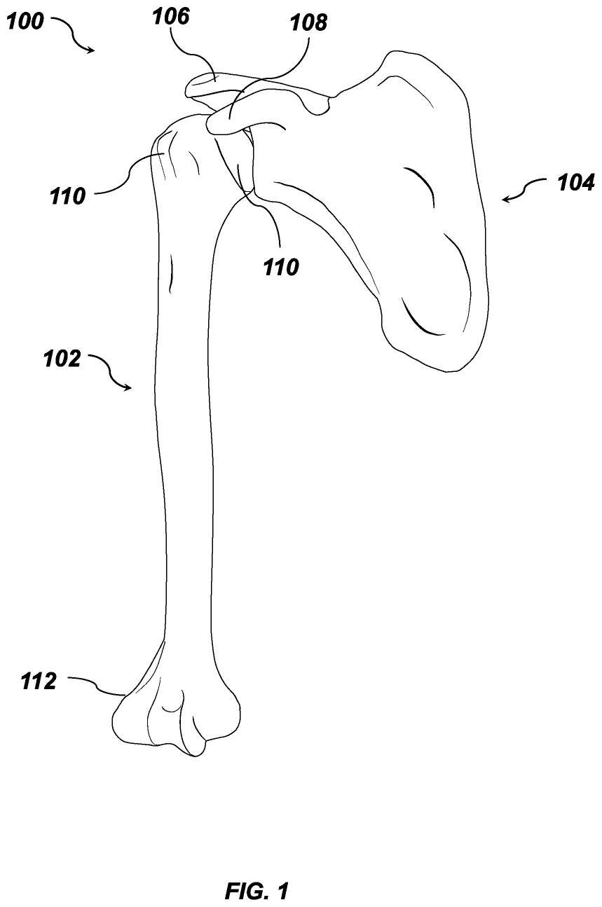

Referring now to FIG. 1, a shoulder joint 100 is shown including a humerus 102, scapula 104, acromion 106, coracoid 108, humeral head 110, and distal humerus 112.

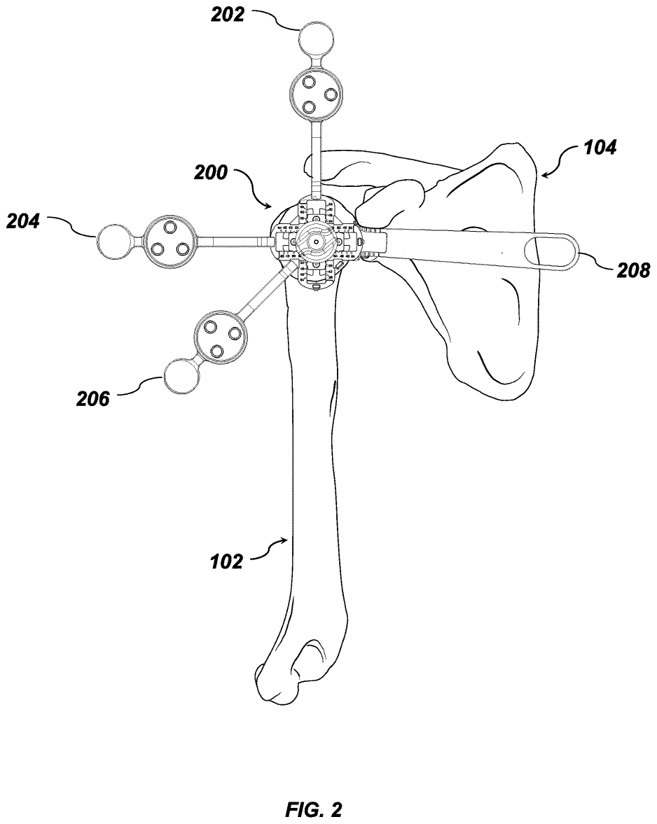

As shown in FIG. 2, a humeral head centering jig 200 (e.g., humeral head surgical guide, surgical guide, etc.) is positioned on the humeral head 110 of the humerus 102. The humeral head centering jig 200 includes a first axis tracking marker 202, a second axis tracking marker 204, a main body tracking marker 206, and humeral head retractor 208 (e.g., mover, manipulator, etc.). The tracking markers (e.g., first axis tracking marker 202, second axis tracking marker 204, main body tracking marker 206) extend radially from the humeral head centering jig 200 and reference specific anatomical landmarks on the proximal humerus. The anatomical landmarks can include, but are not limited to, an articular surface of the humeral head 110, an anatomical neck of the humerus 102, a greater tubercle of the humerus 102, a lesser tubercle of the humerus 102, or an intertubercular groove of the humerus 102 (e.g., between the greater tubercle and the lesser tubercle).

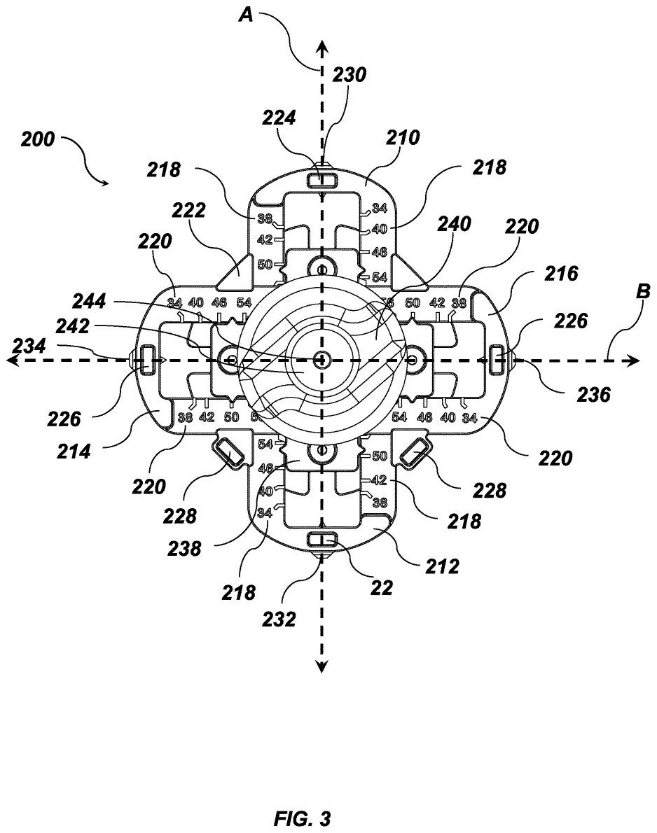

Referring to FIG. 3, a top view of the humeral head centering jig 200 is shown. The jig includes first slider 210 along the first axis, second slider 212 along the first axis, third slider 214 along a second axis, and fourth slider 216 along the second axis. The humeral head diameter size can be measured along the first axis 218 and second axis 220. The humeral head centering jig 200 can include a main humeral head centering jig body 222 that can connect to a non-movable tracking marker via a tracking marker connecting feature 228, and for the movable axes A and B, axis 1 connecting feature 224 and axis 2 connecting feature 226, respectively. Each slider can have a corresponding securing arm: securing arm 230 for slider 210, securing arm 232 for slider 212, securing arm 234 for slider 214, and securing arm 236 for slider 216. A top securing cap 238 can be positioned at the top of the jig 200. Additional features of the humeral head centering jig 200 can include an actuation knob 240, humeral head thickness size guide 242, and shaft for guide pin 244. The humeral head centering jig 200 can establish accurate alignment along axes A and B as shown by the dashed lines, which indicate the primary alignment references for proper positioning of the centering jig.

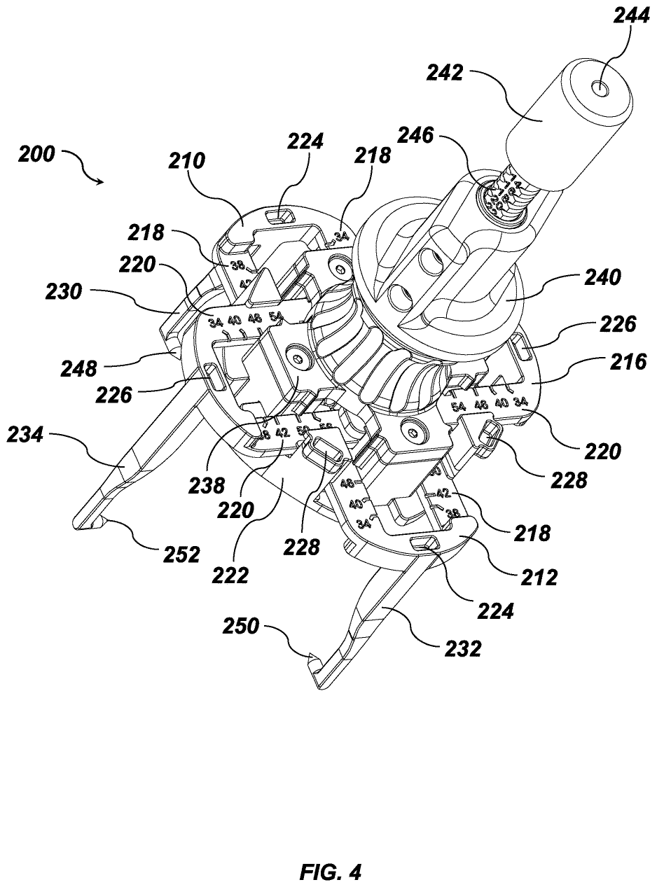

FIG. 4 shows a top perspective view of the humeral head centering jig 200 including a humeral head thickness size 246 and spikes for the securing arms (e.g., securing arm 230, 232, 234, and 236): spike 248 for securing arm 210, spike 250 for securing arm 212, and spike 252 for securing arm 214.

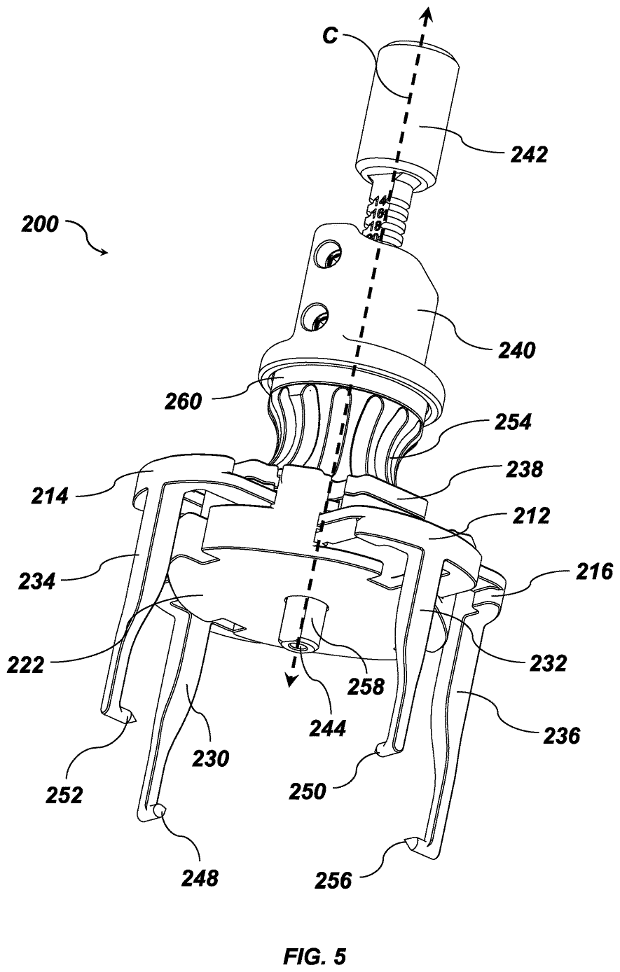

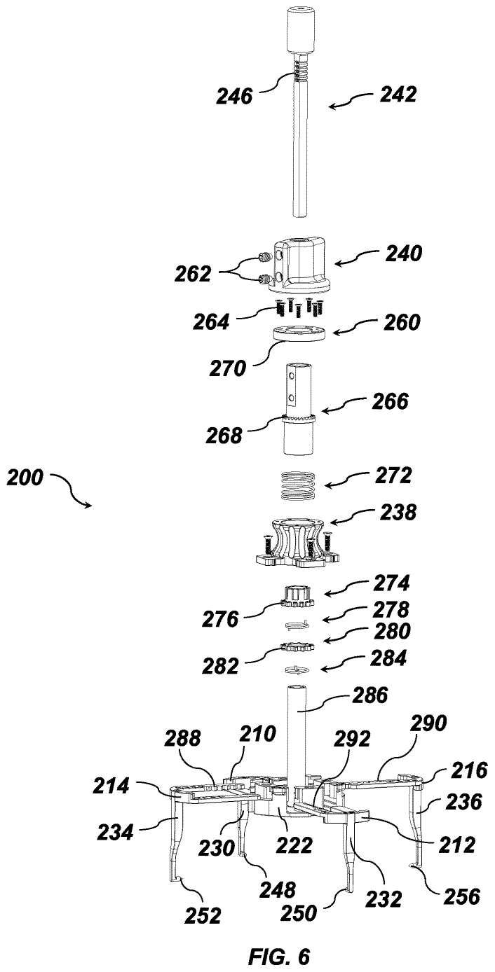

FIG. 5 illustrates a bottom perspective view of the humeral head centering jig 200, showing the finger grip 254 on top securing cap 238 and spike 256 for securing arm 216. The humeral head thickness size guide, distal end 258 is also visible, along with the top securing cap washer 260. The vertical adjustment mechanism can move along axis C as indicated by the dashed line, providing precise control over the depth and orientation of a guide pin. The actuation knob 240 can remain locked in the top position by a spring along axis C, and unlocked by pressing down on the knob to overcome the spring tension. While in the unlocked position, actuation knob 240 can move the sliders 210, 212, 214, and 216 towards the center of the main humeral head centering jig body 222 in response to being turned clockwise and moves the sliders away in response to being turned counterclockwise. To accommodate an axis distance that is not equal to the other axis distance, the mechanism shown in the following FIG. 6 shows a spring mechanism that allows one of the axes to remain stationary while the other axis continues to move while turning the actuation knob 240. The spring mechanism can allow the actuation knob 240 to be tensioned counterclockwise to mitigate loss of tension.

As shown in FIG. 6, an exploded view of the humeral head centering jig 200 reveals the individual components of the humeral head centering jig 200 in greater detail. The humeral head centering jig 200 can include screws 262 to secure actuation knob 240, screws 264 to secure top securing cap washer 260, intermediate shaft 266 with gear teeth 268, gear teeth 270 that interface with gear teeth 268 (not shown), compression spring 272, bottom shaft 274 with gear teeth 276, top torsion spring 278, bottom gear 280 with gear teeth 282, bottom torsion spring 284, and shaft 286 on main body 222. Additional gear teeth can include gear teeth 288 on slider 214, gear teeth 290 on slider 216, and gear teeth 292 on slider 212.

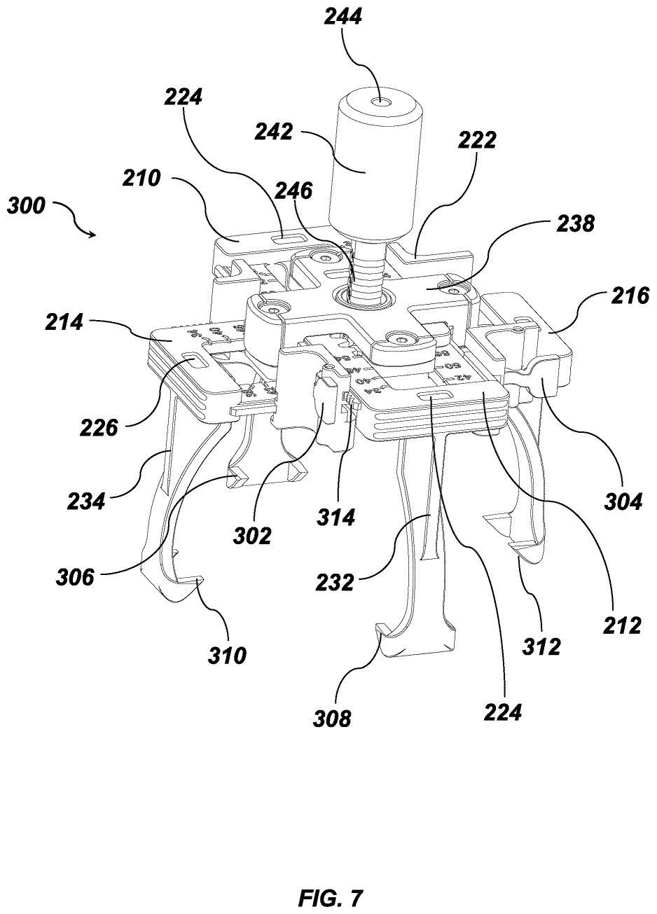

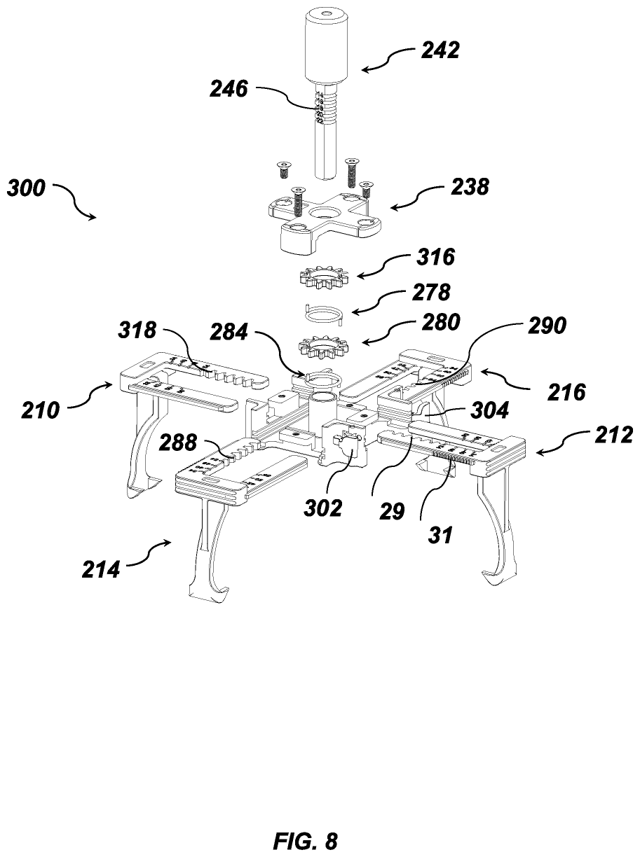

FIGS. 7 and 8 illustrate a humeral head centering jig, multi-spike version 300. In FIG. 7, this alternative centering jig 300 can include a first release button 302 along the first axis, a second release button 304 along the second axis, and multi-spikes for each securing arm including multi-spike 306 for securing arm 210, multi-spike 308 for securing arm 212, multi-spike 310 for securing arm 214, and multi-spike 312 for securing arm 216. Gear teeth 314 on slider 212 can interface with release button 302. FIG. 8 shows an exploded view of the humeral head centering jig 300, revealing additional components including top gear 316 and gear teeth 318 on slider 212.

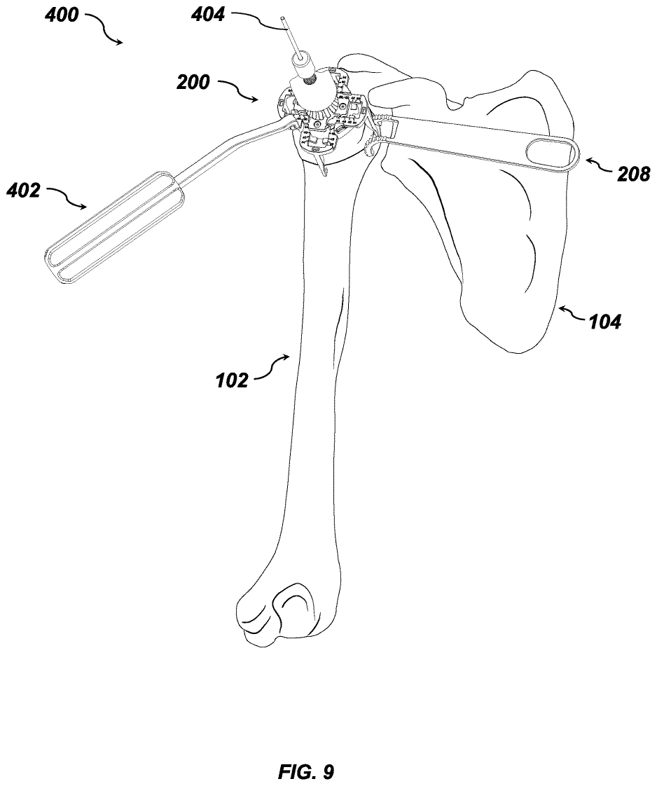

FIG. 9 shows a handle configuration 400 for the humeral head centering jig 200 or 300, featuring a manipulating handle 402, humeral head retractor 208, and guide pin 404. The manipulating handle 402 can facilitate positioning of the centering jig 200 or 300 on the humeral head 110.

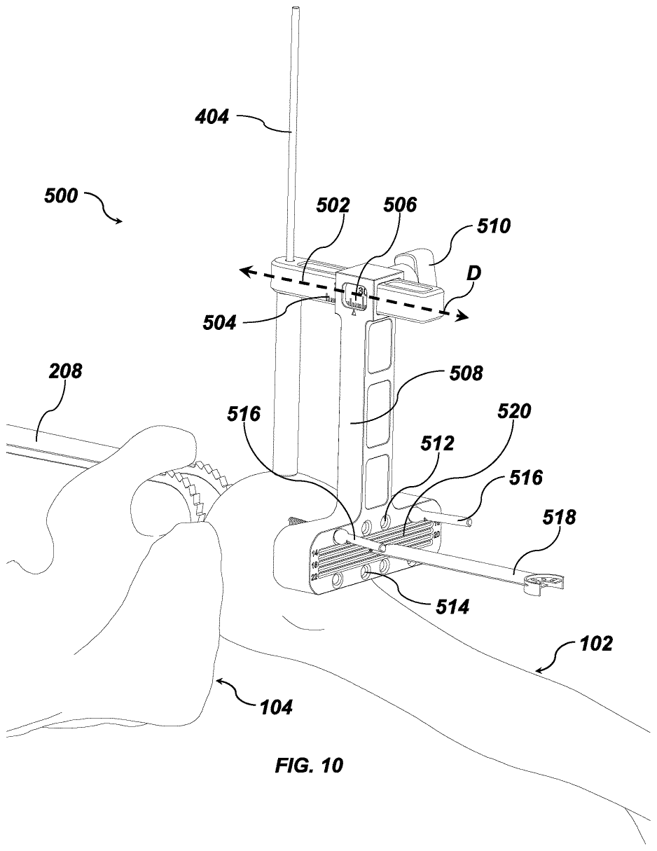

FIG. 10 illustrates a fixed humeral head cutting jig 500 (e.g., surgical apparatus, etc.) connected to the humeral head 110. The fixed cutting jig 500 can include a main fixed cutting jig body 502, measurement tick marks 504, measurement window 506, and adjustable cutting arm 508 with tightening knob 510. The cutting jig 500 can include top holes 512 and bottom holes 514 for olive pins 516 to guide an oscillating saw blade 518 through humeral head thickness slots 520. The main fixed cutting jig body 502 can move along axis D as indicated by the dashed line, allowing precise positioning of the cutting plane.

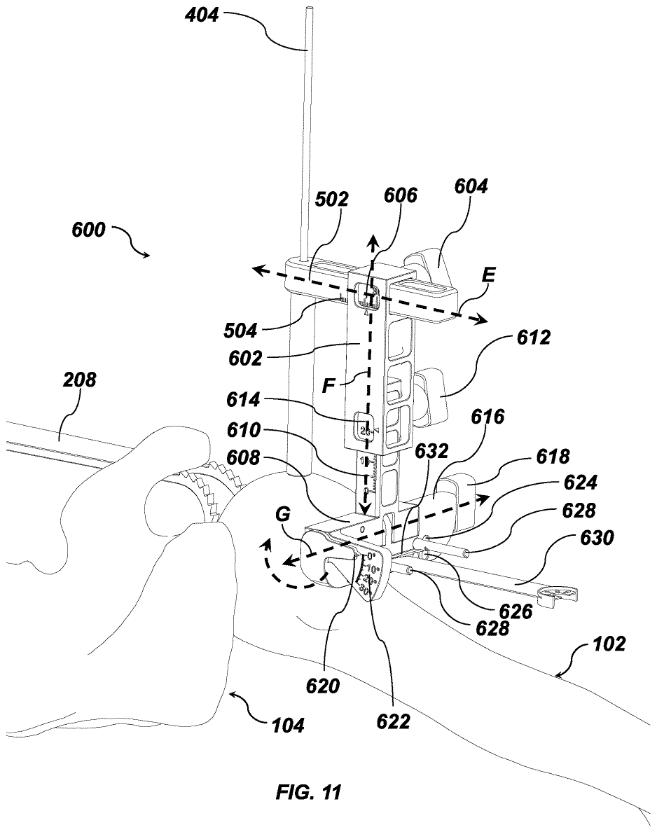

FIG. 11 shows an adjustable humeral head cutting jig 600 connected to the shoulder joint. The adjustable cutting jig 600 can include a first adjustable arm 602 along a first axis with tightening knob 604 and measurement window 606. The adjustable cutting jig 600 can include a second adjustable arm 608 along a second axis with measurement tick marks 610, tightening knob 612, and measurement window 614. The adjustable cutting jig 600 can include a third adjustable arm 616 along a third axis which can include a tightening knob 618, measurement arm 620, and measurement tick marks 622. The cutting jig 600 can include top holes 624 and bottom holes 626 for olive pins 628 to guide an oscillating saw blade 630 through the humeral head cutting slot 632. The cutting jig 600 can move along multiple axes including horizontal axis E, vertical axis F and angular adjustment axis G as indicated by the dashed lines, providing comprehensive control over the cutting plane orientation.

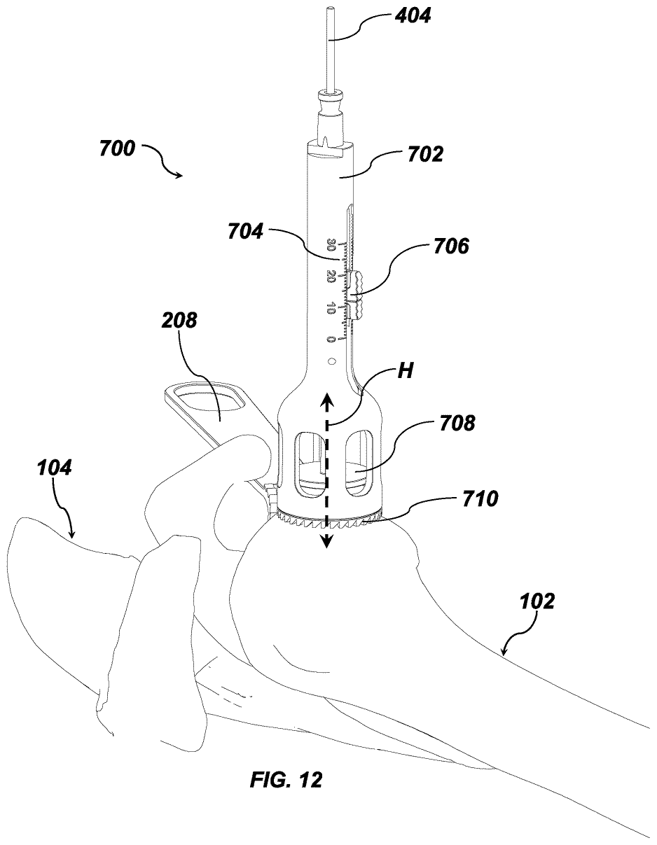

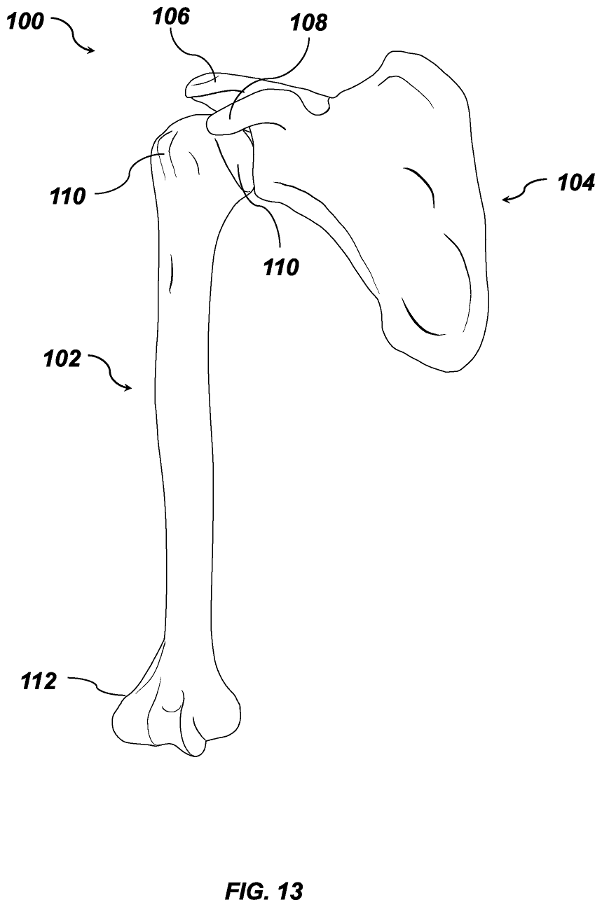

FIGS. 12 and 13 illustrate a humeral head coring drill 700 (e.g., surgical apparatus, etc.) for use with the centering jig 200 or 300. In FIG. 12, the humeral head coring drill 700 can include a coring drill main body 702, measurement tick marks 704 for coring depth, adjusting button 706, coring drill plunger 708, and modular serrated insert 710. FIG. 13 shows an exploded view of the humeral head coring drill 700, revealing additional components including a drill handpiece attachment feature 712, internal threads 714 in main body, serrated edge 716 on insert, external threads 718 on modular serrated insert, and bone core ejection plunger 720.

In operation, the systems and methods of the present disclosure provide a comprehensive system for precise bone preparation during shoulder arthroplasty procedures. The humeral head centering jig (either 200 or multi-spike 300) is first positioned on the humeral head 110 with reference to specific anatomical landmarks, possibly using the handle configuration 400. The centering jig 200 or 300 is then secured to the bone using the various securing arms and spikes. For resection of the humeral head, either the fixed humeral head cutting jig 500 or the adjustable humeral head cutting jig 600 is used, depending on the specific constraints of the procedure. Each cutting jig can provide precise guidance for the oscillating saw blade to make accurate resections. Finally, the humeral head coring drill 700 is used to prepare the central portion of the humeral head. The system allows for precise adjustment of resection angle, depth, and position to accommodate patient-specific anatomy and implant requirements.

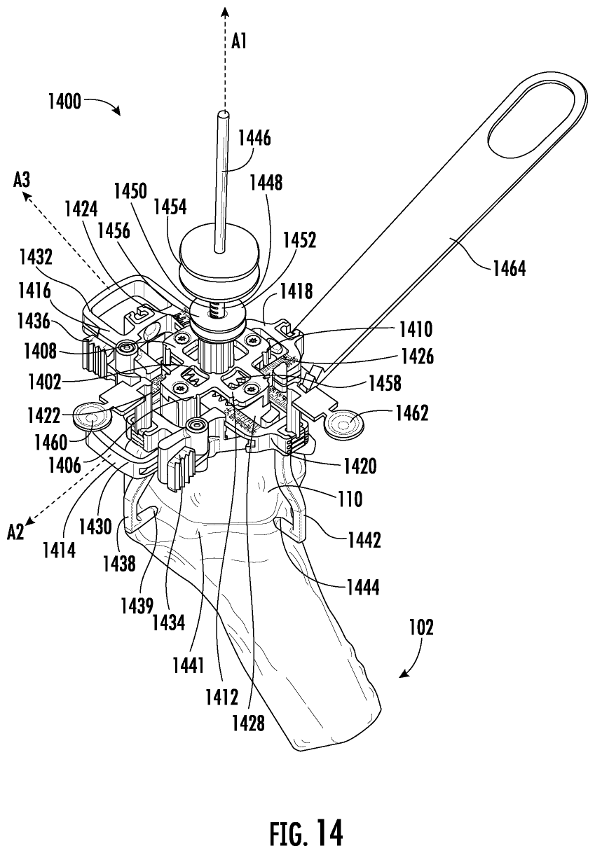

FIGS. 14-17 depict various views of an example humeral head surgical guide 1400 (e.g., centering guide, surgical guide, alignment jig, etc.). The humeral head surgical guide 1400 can be an example of the centering jig 200 or 300. The humeral head surgical guide 1400 can be used to determine dimensions, such as a width, thickness, ellipticity across planes, and length, of a humeral head 110. The humeral head surgical guide 1400 can be used to determine a center of the humeral head 110. In some implementations, the center of the humeral head 110 corresponds to an apex of the humeral head 110. The humeral head surgical guide 1400 can be formed from one or more materials, such as but not limited to metal (e.g., steel, etc.), composites, ceramics, or polymers, among others.

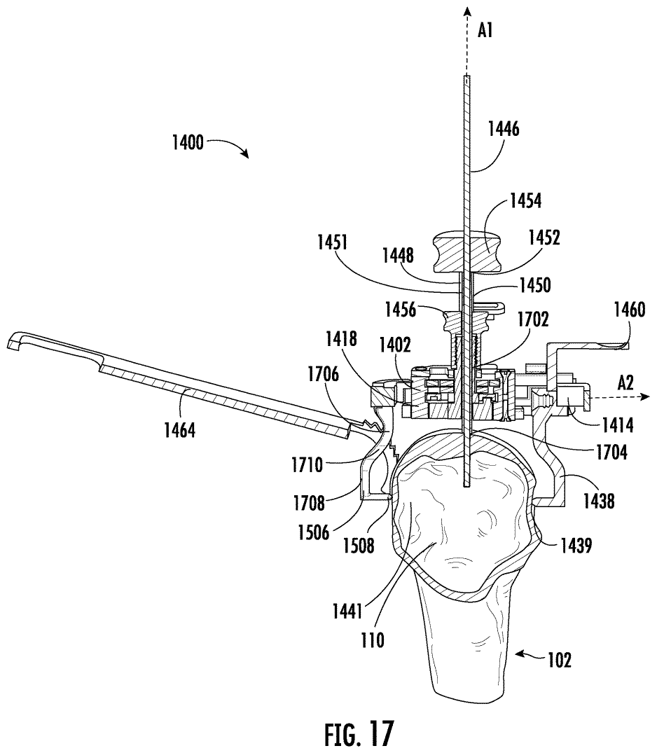

The humeral head surgical guide 1400 can include at least one guide body 1402 (e.g., main body, central body, housing, etc.). The guide body 1402 can be an example of the jig body 222. As shown in at least FIG. 17, the guide body 1402 can define a guide aperture 1702 (e.g., guide hole, central aperture, through-bore, etc.) extending along a first axis A1. The first axis A1 can be at a center of the guide body 1402, such that the guide aperture 1702 extends through the center of the guide body 1402 along the first axis A1. In response to determining the center of the humeral head 110, the guide aperture 1702 can align with the center of the humeral head 110.

The guide body 1402 can include one or more markers. For example, the guide body 1402 can include at least one of a first marker 1406 (e.g., indicator, reference marker, etc.), a second marker 1408, a third marker 1410, and a fourth marker 1412. The first marker 1406, the second marker 1408, the third marker 1410, and the fourth marker 1412 can be used to indicate and determine one or more dimensions of the humeral head 110. The first marker 1406, the second marker 1408, the third marker 1410, and the fourth marker 1412 can have a shape such as but not limited to a triangular, rectangular, circular, or any other shape.

The humeral head surgical guide 1400 can include one or more movable guide members. The humeral head surgical guide 1400 can include at least one of a first movable guide member 1414 (e.g., slider, movable arm, adjustable member, etc.), a second movable guide member 1416, a third movable guide member 1418, or a fourth movable guide member 1420. For example, in some implementations, the humeral head surgical guide 1400 includes the first movable guide member 1414 and the second movable guide member 1416 and in other implementations, the humeral head surgical guide 1400 includes the first movable guide member 1414, the second movable guide member 1416, the third movable guide member 1418, and the fourth movable guide member 1420. The first movable guide member 1414 can be an example of slider 210. The second movable guide member 1416 can be an example of slider 214. The third movable guide member 1418 can be an example of slider 212. The fourth movable guide member 1420 can be an example of slider 216.

At least one of the first movable guide member 1414, the second movable guide member 1416, the third movable guide member 1418, or the fourth movable guide member 1420 can be coupled to the guide body 1402. For example, the first movable guide member 1414, the second movable guide member 1416, the third movable guide member 1418, and the fourth movable guide member 1420 can be coupled to the guide body 1402 and extend from the guide body 1402. In some implementations, the first movable guide member 1414 is coupled to the third movable guide member 1418 and the second movable guide member 1416 is coupled to the fourth movable guide member 1420. In some implementations, at least one of the first movable guide member 1414, the second movable guide member 1416, the third movable guide member 1418, or the fourth movable guide member 1420 is in contact with each other.

Each of the first movable guide member 1414, the second movable guide member 1416, the third movable guide member 1418, and the fourth movable guide member 1420 can extend and move along an axis relative to the guide body 1402. For example, the first movable guide member 1414 can extend and move along a second axis A2 perpendicular to the first axis A1. The second axis A2 can be an example of the axis A. As another example, the second movable guide member 1416 can extend and move along a third axis A3 perpendicular to the first axis A1 and relative to the second axis A2. The third axis A3 can be an example of the axis B. In some implementations, the third axis A3 is perpendicular to the first axis A1 and the second axis A2. In other implementations, the third axis A3 can intersect the second axis A2 at an angle other than 90 degrees, such as less than 90 degrees.

In implementations where the humeral head surgical guide 1400 includes the third movable guide member 1418 and the fourth movable guide member 1420, the third movable guide member 1418 can extend and move along the second axis A2. Consequently, the third movable guide member 1418 can be aligned with the first movable guide member 1414 and in some implementations, the first movable guide member 1414 and the third movable guide member 1418 can be centered on the second axis A2. The fourth movable guide member 1420 can extend and move along the third axis A3. Consequently, the fourth movable guide member 1420 can be aligned with the second movable guide member 1416 and in some implementations, the second movable guide member 1416 and the fourth movable guide member 1420 can be centered on the third axis A3.

The first movable guide member 1414, the second movable guide member 1416, the third movable guide member 1418, and the fourth movable guide member 1420 can each include indicators. For example, the first movable guide member 1414 can include one or more first indicators 1422, the second movable guide member 1416 can include one or more second indicators 1424, the third movable guide member 1418 can include one or more third indicators 1426, and the fourth movable guide member 1420 can include one or more fourth indicators 1428. The indicators (e.g., first indicators 1422, second indicators 1424, third indicators 1426, fourth indicators 1428) can be disposed along a length of the movable guide members (e.g., first movable guide member 1414, second movable guide member 1416, third movable guide member 1418, fourth movable guide member 1420), and can indicate various measurement values that correspond to dimensions (e.g., width, length, ellipticity, etc.) of the humeral head 110. In some implementations, each of the movable guide members can have a length between 30 to 60 millimeters (mm), inclusive. In some implementations, the length of each of the movable guide members is less than 30 or greater than 60 mm.

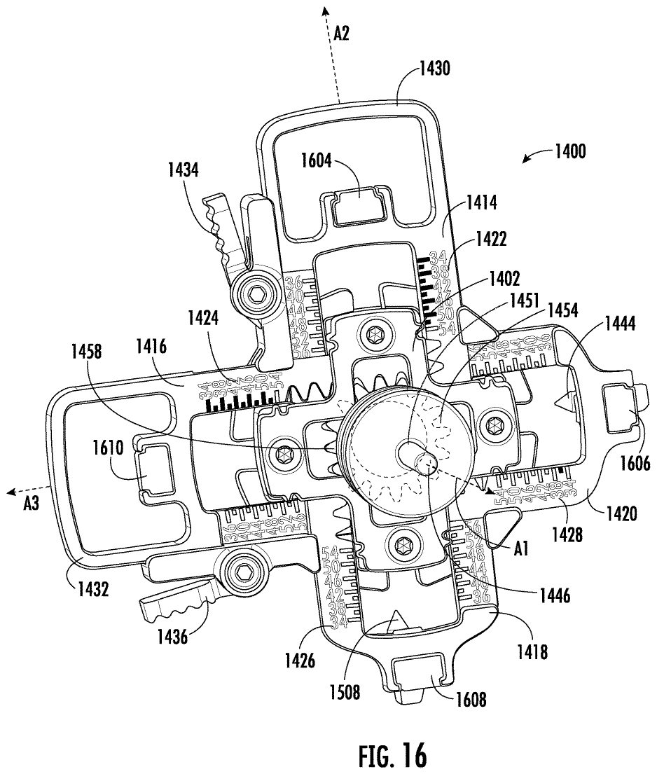

The markers (e.g., first marker 1406, second marker 1408, third marker 1410, fourth marker 1412) can be oriented relative or towards the indicators. For example, the markers can include at least one point, and the point can face and be directed towards the indicators to indicate which indicator corresponds to a dimension of the humeral head 110. Alignment of the marker with at least one indicator can determine a respective dimension of the humeral head 110. In some implementations, the markers can be located above the indicators relative to the guide body 1402. For example, the first marker 1406 can have a triangle shape, and one point of the first marker 1406 can face toward the first indicators 1422. In some implementations, as shown in FIG. 16, the guide body 1402 can include one marker per set of indicators. For example, in some implementations, each of the movable guide members can include two sets of indicators along opposing lengths (e.g., portions, etc.) of the movable guide members. Consequently, the guide body 1402 can include a marker per set of indicators.

In some implementations, only the first movable guide member 1414 or the second movable guide member 1416 includes indicators. In other implementations, only the third movable guide member 1418 and the fourth movable guide member 1420 includes indicators. The first movable guide member 1414 and the third movable guide member 1418 can indicate a dimension different from a dimension indicated by the second movable guide member 1416 and the fourth movable guide member 1420.

In some implementations, the humeral head surgical guide 1400 can include one or more handles to move one or more movable guide members. For example, the humeral head surgical guide 1400 can include a first handle 1430 coupled to the first movable guide member 1414 and a second handle 1432 coupled to the second movable guide member 1416. A user, such as a clinician or surgeon, can contact and apply a force to the first handle 1430 and the second handle 1432 to move the first movable guide member 1414 and the second movable guide member 1416, respectively. In some implementations, the first handle 1430 and the second handle 1432 can be used to move the guide body 1402. In some implementations, the humeral head surgical guide 1400 includes handles coupled to at least one of the third movable guide member 1418 and or fourth movable guide member 1420.

In implementations where the humeral head surgical guide 1400 includes one or more handles coupled to one or more movable guide members, the humeral head surgical guide 1400 can further include one or more actuators (e.g., clamps, etc.). For example, the humeral head surgical guide 1400 can include a first actuator 1434 coupled to at least one of the first movable guide member 1414 or the guide body 1402, and a second actuator 1436 coupled to at least one of the second movable guide member 1416 or the guide body 1402. The first actuator 1434 can allow or stop movement of the first movable guide member 1414, and the second actuator 1436 can allow or stop movement of the second movable guide member 1416. For example, in response to being actuated, the first actuator 1434 and the second actuator 1436 can stop movement of the first movable guide member 1414 and second movable guide member 1416, respectively, by, for example, engaging with the guide body 1402. In response to being released, the first actuator 1434 and the second actuator 1436 can allow movement of the first movable guide member 1414 and second movable guide member 1416, respectively.

In some implementations, the first actuator 1434 and the second actuator 1436 can stop and allow movement of the third movable guide member 1418 and the fourth movable guide member 1420, respectively. For example, actuation of the first actuator 1434 can stop movement for both the first movable guide member 1414 and the third movable guide member 1418. In some implementations, the first handle 1430 can be used to move at least one of the first movable guide member 1414 or the third movable guide member 1418. In some implementations, the second handle 1432 can be used to move at least one of the second movable guide member 1416 or the fourth movable guide member 1420. In some implementations, the first actuator 1434 and the second actuator 1436 are removably coupled from the first movable guide member 1414 and the second movable guide member 1416, respectively, or the guide body 1402.

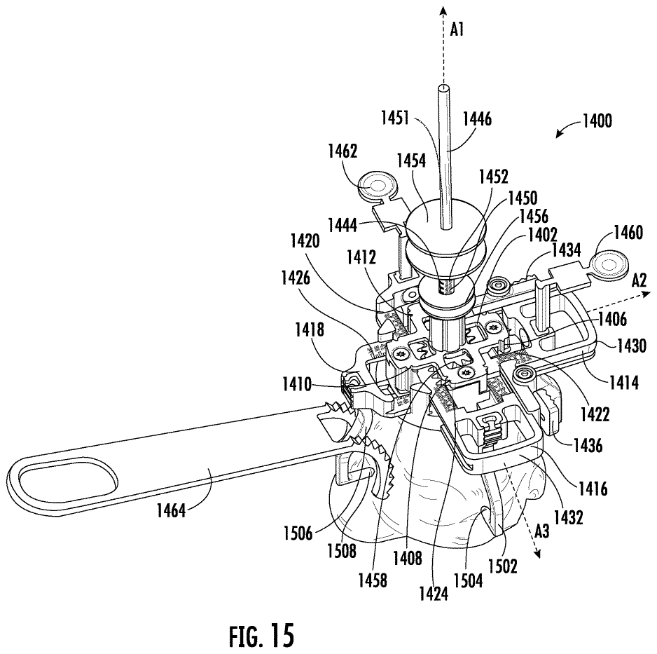

As shown in FIGS. 14-15, the humeral head surgical guide 1400 can include one or more supporting members. Each of the supporting members can include at least one engagement feature (e.g., tooth, spike, etc.) to engage with and contact the humeral head 110. The humeral head surgical guide 1400 can include a first supporting member 1438 (e.g., support arm, etc.) coupled to and extending from the first movable guide member 1414. For example, the first supporting member 1438 can extend below the first movable guide member 1414. The first supporting member 1438 can include at least one first engagement feature 1439. The first engagement feature 1439 can face and extend towards the first axis A1. The first engagement feature 1439 can contact a bottom surface 1441 of the humeral head 110. For example, the first movable guide member 1414 can be adjusted along the second axis A2 such that the first engagement feature 1439 contacts and engages with the bottom surface 1441 to at least provide stability and support to the humeral head surgical guide 1400.

The first marker 1406 can be oriented relative to the one or more first indicators 1422 to indicate a first distance between the first axis A1 and the first engagement feature 1439. For example, the first distance can represent a dimension of the humeral head 110, such as a first portion of a width of the humeral head 110. The first movable guide member 1414 can be moved using, for example, the first handle 1430 such that the first engagement feature 1439 contacts the bottom surface 1441 of the humeral head 110. Upon contact of the first engagement feature 1439 with the bottom surface 1441, the first marker 1406 can be aligned with a first indicator 1422 or between first indicators 1422, and the alignment of the first marker 1406 along the first indicators 1422 can represent the first distance. In some implementations, the first distance represents a dimension of a plane between the humeral head 110 and the humerus 102.

As shown in at least FIG. 15, the humeral head surgical guide 1400 can include a second supporting member 1502 coupled to and extending from the second movable guide member 1416. For example, the second supporting member 1502 can extend below the second movable guide member 1416. The second supporting member 1502 can include at least one second engagement feature 1504. The second engagement feature 1504 can face and extend towards the first axis A1. The second engagement feature 1504 can contact the bottom surface 1441 of the humeral head 110. For example, the second movable guide member 1416 can be adjusted along the third axis A3 such that the second engagement feature 1504 contacts and engages with the bottom surface 1441 to at least provide stability and support to the humeral head surgical guide 1400. The second engagement feature 1504 can contact and engage with a different portion of the bottom surface 1441 from the first engagement feature 1439. In some implementations, the second engagement feature 1504 can extend along an axis parallel to and offset from the third axis A3 and the first engagement feature 1439 can extend along an axis parallel to and offset from the second axis A2.

The second marker 1408 can be oriented relative to the one or more second indicators 1424 to indicate a second distance between the first axis A1 and the second engagement feature 1504. For example, the second distance can represent a dimension of the humeral head 110, such as a first portion of a length of the humeral head 110. The second movable guide member 1416 can be moved using, for example, the second handle 1432 such that the second engagement feature 1504 contacts the bottom surface 1441 of the humeral head 110. Upon contact of the second engagement feature 1504 with the bottom surface 1441, the second marker 1408 can be aligned with a second indicator 1424 or between second indicators 1424, and the alignment of the second marker 1408 along the second indicators 1424 can represent the second distance. In some implementations, the second distance represents a dimension of a plane between the humeral head 110 and the humerus 102.

The humeral head surgical guide 1400 can include a third supporting member 1506 coupled to and extending from the third movable guide member 1418. For example, the third supporting member 1506 can extend below the third movable guide member 1418. As shown in at least FIG. 16, the third supporting member 1506 can include at least one third engagement feature 1508. The third engagement feature 1508 can face and extend towards the first axis A1. The third engagement feature 1508 can contact the bottom surface 1441 of the humeral head 110. The third engagement feature 1508 can contact and engage with a different portion of the bottom surface 1441 from the first engagement feature 1439 and the second engagement feature 1504. For example, the third movable guide member 1418 can be adjusted along the second axis A2 such that the third engagement feature 1508 contacts and engages with the bottom surface 1441 to at least provide stability and support to the humeral head surgical guide 1400.

The third marker 1410 can be oriented relative to the one or more third indicators 1426 to indicate a third distance between the first axis A1 and the third engagement feature 1508. For example, the third distance can represent a dimension of the humeral head 110, such as a second portion of a width of the humeral head 110. In some implementations, a combination of the first distance and the third distance can represent a width of the humeral head 110. The third movable guide member 1418 can be moved such that the third engagement feature 1508 contacts the bottom surface 1441 of the humeral head 110. Upon contact of the third engagement feature 1508 with the bottom surface 1441, the third marker 1410 can be aligned with a third indicator 1426 or between third indicators 1426, and the alignment of the third marker 1410 along the third indicators 1426 can represent the third distance. In some implementations, the third distance represents a dimension of a plane between the humeral head 110 and the humerus 102. In some implementations, the third distance can be same as the first distance. For example, the first marker 1406 and the third marker 1410 can indicate a same value for a dimension, such as a width or length of the humeral head 110.

Referring back to FIG. 14, the humeral head surgical guide 1400 can include a fourth supporting member 1442 coupled to and extending from the fourth movable guide member 1420. For example, the fourth supporting member 1442 can extend below the fourth movable guide member 1420. The fourth supporting member 1442 can include at least one fourth engagement feature 1444. The fourth engagement feature 1444 can face and extend towards the first axis A1. The fourth engagement feature 1444 can contact the bottom surface 1441 of the humeral head 110. For example, the fourth movable guide member 1420 can be adjusted along the third axis A3 such that the fourth engagement feature 1444 contacts and engages with the bottom surface 1441 to at least provide stability and support to the humeral head surgical guide 1400.

The fourth marker 1412 can be oriented relative to the one or more fourth indicators 1428 to indicate a fourth distance between the first axis A1 and the fourth engagement feature 1444. For example, the fourth distance can represent a dimension of the humeral head 110, such as a second portion of a length of the humeral head 110. In some implementations, a combination of the second distance and the fourth distance can represent a length of the humeral head 110. The fourth movable guide member 1420 can be moved such that the fourth engagement feature 1444 contacts the bottom surface 1441 of the humeral head 110. Upon contact of the fourth engagement feature 1444 with the bottom surface 1441, the fourth marker 1412 can be aligned with a fourth indicator 1428 or between fourth indicators 1428, and the alignment of the fourth marker 1412 along the fourth indicators 1428 can represent the fourth distance. In some implementations, the fourth distance represents a dimension of a plane between the humeral head 110 and the humerus 102. The fourth engagement feature 1444 can contact and engage with a different portion of the bottom surface 1441 from the first engagement feature 1439, the second engagement feature 1504, and the third engagement feature 1508. In some implementations, the fourth engagement feature 1444 can extend along an axis parallel to and offset from the third axis A3 and the third engagement feature 1508 can extend along an axis parallel to and offset from the second axis A2. In some implementations, the second distance can be same as the fourth distance. For example, the second marker 1408 and the fourth marker 1412 can indicate a same value for a dimension, such as a width or length of the humeral head 110.

In some implementations, the supporting members (e.g., first supporting member 1438, second supporting member 1502, third supporting member 1506, fourth supporting member 1442) are removably coupled to the respective movable guide members. For example, the supporting members can be screwed into respective movable guide members. In implementations where the supporting members are removably coupled to the respective movable guide members, the supporting members can be removed and swapped with supporting members of different dimensions. In some implementations, the supporting members and engagement features (e.g., first engagement feature 1439, second engagement feature 1504, third engagement feature 1508, fourth engagement feature 1444) can secure and provide stability to the humeral head surgical guide 1400 on the humeral head 110. At least one of the first distance, the second distance, the third distance, or the fourth distance can be used to determine dimensions of the humeral head 110 and to select an implant corresponding to the dimensions. In some implementations, each of the supporting members have a length between 20 to 60 mm, inclusive. In some implementations, the length of each of the supporting members have a length less than 20 or greater than 60 mm. In some implementations, the supporting members are removably coupled from the guide body 1402 and in other implementations, the supporting members are integral with and form a monolithic apparatus with the guide body 1402.

The guide body 1402 can receive a guide wire 1446 (e.g., guide pin, reference wire, etc.) that can extend through the guide aperture 1702. The guide wire 1446 can be formed from a material including but not limited to stainless steel, titanium, or any other material. The guide wire 1446 can have a diameter less than the guide aperture 1702, and can be inserted into the guide body 1402 via the guide aperture 1702. The guide aperture 1702 can extend along an entire length or height of the guide body 1402. The guide wire 1446, once inserted, can extend through and from the guide aperture 1702 on both sides of the guide body 1402. The guide wire 1446 can be attachable to the center of the humeral head 110 using at least the first movable guide member 1414, the first supporting member 1438, the second supporting member 1502, and the second movable guide member 1416. For example, the at least one of the first movable guide member 1414 or the second movable guide member 1416 can be adjusted such that the first engagement feature 1439 and the second engagement feature 1504, respectively, contact and engage with the humeral head 110 and to determine the center of the humeral head 110. Following the determination of the center, the guide wire 1446 can be drilled into the center of the humeral head 110 to attach the guide wire 1446 to the center of the humeral head 110. For example, the guide wire 1446 can be attached to a drill once inserted through the guide aperture 1702 to be drilled into and attached to the center of the humeral head 110.

Movement of at least one of the first movable guide member 1414, second movable guide member 1416, third movable guide member 1418, or fourth movable guide member 1420 can determine the center of the humeral head 110. For example, in response to the humeral head surgical guide 1400 being placed in contact with the humeral head 110, the at least one of the first movable guide member 1414, second movable guide member 1416, third movable guide member 1418, or fourth movable guide member 1420 can be moved and adjusted until at least one of the first engagement feature 1439, second engagement feature 1504, third engagement feature 1508, or fourth engagement feature 1444 contact the bottom surface 1441. The humeral head surgical guide 1400 can be centered via adjustment of the at least one of the first movable guide member 1414, second movable guide member 1416, third movable guide member 1418, or fourth movable guide member 1420, such as by each of the first engagement feature 1439, second engagement feature 1504, third engagement feature 1508, and fourth engagement feature 1444 engaging with the bottom surface 1441. Consequently, once adjusted, the guide aperture 1702 can align with the center of the humeral head 110. Then, the guide wire 1446 can be extended through the guide aperture 1702 and attached to the center of the humeral head 110. Once attached, the guide wire 1446 can be used as a reference point for preparing the humeral head 110 for receiving an implant (e.g., shoulder or humeral head implant, etc.).

In some implementations, the first actuator 1434 and the second actuator 1436 release the first movable guide member 1414, second movable guide member 1416, the third movable guide member 1418, and the fourth movable guide member 1420. For example, the first movable guide member 1414, second movable guide member 1416, the third movable guide member 1418, and the fourth movable guide member 1420 are adjusted such that respective engagement features engage with the humeral head 110. After attaching the guide wire 1446 to the humeral head 110, the humeral head surgical guide 1400 can be removed, such as by actuating the first actuator 1434 and the second actuator 1436 which moves the first movable guide member 1414, second movable guide member 1416, the third movable guide member 1418, and the fourth movable guide member 1420 back to an original position (e.g., prior to adjustment, etc.) away from the humeral head 110.

In some implementations, the humeral head surgical guide 1400 can include at least one shaft 1448 (e.g., rod, post, etc.). The shaft 1448 can be coupled to and extend through and from the guide body 1402. For example, the shaft 1448 can extend from both sides of the guide body 1402. The shaft 1448 can define a shaft aperture 1451. The shaft aperture 1451 can be aligned with the guide aperture 1702 such that the guide wire 1446 extends through both the shaft aperture 1451 and the guide aperture 1702 to attach to the center of the humeral head 110. Consequently, the shaft aperture 1451 can extend along the first axis A1. In some implementations, the guide wire 1446 extends through the shaft aperture 1451, and the shaft 1448 can extend through the guide aperture 1702.

As shown in at least FIG. 14, the shaft 1448 can include one or more fifth indicators 1450. The fifth indicators 1450 can be located along a length of the shaft 1448. The shaft 1448 can include one or more ends. As shown in at least FIG. 17, the shaft 1448 can include a first end 1452 and second end 1704 opposite the first end 1452. The fifth indicators 1450 can indicate a fifth distance between the second end 1704 and at least one of the at least one first engagement feature 1439 or second engagement feature 1504. The fifth distance can represent a depth or thickness of the humeral head 110. The fifth indicators 1450 can represent the fifth distance in response to the second end 1704 being in contact with a surface (e.g., top surface) opposite the bottom surface 1441 of the humeral head 110.

In some implementations, the humeral head surgical guide 1400 can include a third handle 1454 coupled to and extending around the shaft 1448. The third handle 1454 can be used to move the shaft 1448 along the first axis A1 to contact the second end 1704 with the humeral head 110 to determine the depth of the humeral head 110 from the fifth indicators 1450. In some implementations, the third handle 1454 defines a handle aperture extending along the first axis A1, and the guide wire 1446 extends through the handle aperture. In some implementations, the humeral head surgical guide 1400 can include a fifth marker 1456 coupled to at least one of the guide body 1402 or the shaft 1448, and can be used to indicate the fifth distance. For example, the third handle 1454 can move the shaft 1448 along with the fifth indicators 1450 along the first axis A1, and upon the second end 1704 contacting the humeral head 110 or the humeral head 110, the fifth marker 1456 can align with a fifth indicator 1450 or between fifth indicators 1450, and the alignment of the fifth marker 1456 with the fifth indicators 1450 can be used to determine the fifth distance. In some implementations, the shaft 1448 can include one or more threads, and rotation of the third handle 1454 can allow the shaft 1448 to move along the first axis A1 and the guide wire 1446 via the one or more threads.

In some implementations, the humeral head surgical guide 1400 includes at least one gear 1458. The gear 1458 can be centered on the first axis A1 and in some implementations, the guide wire 1446 extends through a center of the gear 1458. In some implementations, the gear 1458 extends around the guide aperture 1702. The gear 1458 can be coupled and engaged with at least the first movable guide member 1414 and the second movable guide member 1416. In some implementations, the gear 1458 is coupled and engaged with the first movable guide member 1414, the second movable guide member 1416, the third movable guide member 1418, and the fourth movable guide member 1420. For example, at least one of the first movable guide member 1414, the second movable guide member 1416, the third movable guide member 1418, or the fourth movable guide member 1420 can partially extend into and contact the gear 1458 such that the gear 1458 is rotatably coupled to the at least one of the first movable guide member 1414, the second movable guide member 1416, the third movable guide member 1418, or the fourth movable guide member 1420.

The gear 1458 can maintain the at least one first engagement feature 1439 and second engagement feature 1504 at a same distance from the first axis A1. For example, in response to the first movable guide member 1414 being moved a distance, the second movable guide member 1416 moves the same distance via the gear 1458 in parallel such that at least one first engagement feature 1439 and second engagement feature 1504 maintain a same distance from the first axis A1. In some implementations, the gear 1458 maintains the first engagement feature 1439, the second engagement feature 1504, the third engagement feature 1508, and the fourth engagement feature 1444 at a same distance from the first axis A1. Consequently, the gear 1458 can maintain a centering and symmetry of the first movable guide member 1414, the second movable guide member 1416, the third movable guide member 1418, and the fourth movable guide member 1420 relative to the first axis A1. Thus, in response to the first engagement feature 1439, the second engagement feature 1504, the third engagement feature 1508, and the fourth engagement feature 1444 contacting and engaging with the bottom surface 1441, a position of the guide aperture 1702 aligns with the center of the humeral head 110. In some implementations, the first distance, the second distance, the third distance, and the fourth distance can be equal to each other.

In some implementations, the first movable guide member 1414 and the second movable guide member 1416 are independently movable. For example, movement of the first movable guide member 1414 may not affect movement of the second movable guide member 1416. In some implementations, at least one of the first movable guide member 1414, the second movable guide member 1416, the third movable guide member 1418, or the fourth movable guide member 1420 are independently movable. For example, movement of one movable guide member may not affect movement of other movable guide members. In some implementations, the first engagement feature 1439, the second engagement feature 1504, the third engagement feature 1508, and the fourth engagement feature 1444 can have different distances (e.g., first, second, third, fourth distance) from the first axis A1.

For example, in response to, for example, the first engagement feature 1439 engaging with and contacting the bottom surface 1441, the humeral head surgical guide 1400 can be adjusted, such as the third movable guide member 1418 moved such that the third engagement feature 1508 contacts and engages with the bottom surface 1441 while maintaining a position of the first movable guide member 1414. Consequently, at least one of the first distance, the second distance, the third distance, or the fourth distance can be different, and movement of the guide members can be independent of each other.

In some implementations, the gear 1458 can cause the first movable guide member 1414 and the second movable guide member 1416 to move at a same rate. The gear 1458 can rotate at a rate as the first movable guide member 1414 and the second movable guide member 1416 move, and the rate at which the gear 1458 rotates can translate to a rate at which the first movable guide member 1414 and the second movable guide member 1416 move along the second axis A2 and the third axis A3, respectively. In some implementations, the gear 1458 can cause the first movable guide member 1414, the second movable guide member 1416, the third movable guide member 1418, and the fourth movable guide member 1420 to move at a same rate.

In some implementations, the humeral head surgical guide 1400 can include one or more extenders. The humeral head surgical guide 1400 can include a first extender 1460 coupled to and extending from the first movable guide member 1414. The first extender 1460 can be an example of the first axis tracking marker 202. The first extender 1460 can be removably attached to the first movable guide member 1414. For example, as shown in FIG. 16, the first movable guide member 1414 can define a first attachment point 1604 (e.g., connector, coupling feature, etc.), which can receive and couple the first extender 1460 to the first movable guide member 1414. The first attachment point 1604 can be an example of connecting feature 224. The first extender 1460 can couple to the first movable guide member 1414 and indicate a first anatomical location of the humeral head 110. For example, the first extender 1460 can indicate at least one of an articular apex, greater tuberosity, lesser tuberosity, anatomic neck, superior articular margin, or inferomedial articular margin of at least one of the humeral head 110 or humerus 102. The user can use the first anatomical location indicated by the first extender 1460 to position the humeral head surgical guide 1400 and move the first movable guide member 1414.

The humeral head surgical guide 1400 can include a second extender 1462 coupled to and extending from the fourth movable guide member 1420. The second extender 1462 can be an example of the second axis tracking marker 204. The second extender 1462 can be removably attached to the fourth movable guide member 1420. For example, as shown in FIG. 16, the fourth movable guide member 1420 can define a second attachment point 1606, which can receive and couple the second extender 1462 to the fourth movable guide member 1420. The second attachment point 1606 can be an example of connecting feature 226. The second extender 1462 can couple to the fourth movable guide member 1420 and indicate a second anatomical location of the humeral head 110. For example, the second extender 1462 can indicate at least one of an articular apex, greater tuberosity, lesser tuberosity, anatomic neck, superior articular margin, or inferomedial articular margin of at least one of the humeral head 110 or humerus 102. The user can use the second anatomical location indicated by the second extender 1462 to position the humeral head surgical guide 1400 and move the fourth movable guide member 1420.

In some implementations, at least one of the first extender 1460 or the second extender 1462 can couple to the third movable guide member 1418 or the second movable guide member 1416. For example, the third movable guide member 1418 can include a third attachment point 1608 and the second movable guide member 1416 can include a fourth attachment point 1610. The first extender 1460 and the second extender 1462 can couple to and extend from two of the first attachment point 1604, the second attachment point 1606, the third attachment point 1608, and the fourth attachment point 1610. In some implementations, the humeral head surgical guide 1400 can include more than two extenders to couple to at least one of the first attachment point 1604, the second attachment point 1606, the third attachment point 1608, or the fourth attachment point 1610.

In some implementations, the humeral head surgical guide 1400 includes at least one manipulator 1464. The manipulator 1464 can be configured to engage with and contact at least one of the guide body 1402 or a movable guide member, such as the third movable guide member 1418 to position and adjust the guide body 1402. For example, the user can use the manipulator 1464 to place the guide body 1402 on the humeral head 110 and to reposition the guide body 1402.

Referring back to FIG. 17, in some implementations, the supporting member (e.g., first supporting member 1438, second supporting member 1502, third supporting member 1506, fourth supporting member 1442) can each include a first linear portion 1706, a second linear portion 1708, and a curved portion 1710 connecting the first linear portion 1706 and the second linear portion 1708. The curved portion 1710 can be contiguous with the first linear portion 1706 and the second linear portion 1708. The first linear portion 1706 can extend along an axis parallel to and offset from an axis the second linear portion 1708 extends along. The engagement feature (e.g., the first engagement feature 1439, the second engagement feature 1504, the third engagement feature 1508, and the fourth engagement feature 1444) can extend from the second linear portion 1708.

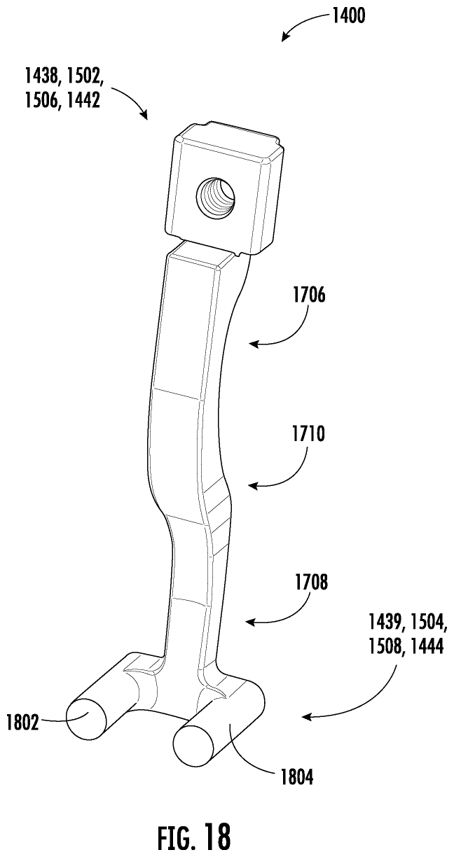

In some implementations, the engagement feature (e.g., the first engagement feature 1439, the second engagement feature 1504, the third engagement feature 1508, and the fourth engagement feature 1444) can include one spike (e.g., tooth, prong, projection, etc.) to contact and engage with the bottom surface 1441. As shown in FIG. 18, in some implementations, the engagement feature can include more than one spike, such as a first spike 1802 and a second spike 1804 that can contact and engage with the bottom surface 1441. The first spike 1802 and the second spike 1804 can be located on a same plane, and can contact different points of the bottom surface 1441 in a same portion. In some implementations, the engagement feature can facilitate stabilization and engage the guide body 1402 with the humeral head 110.

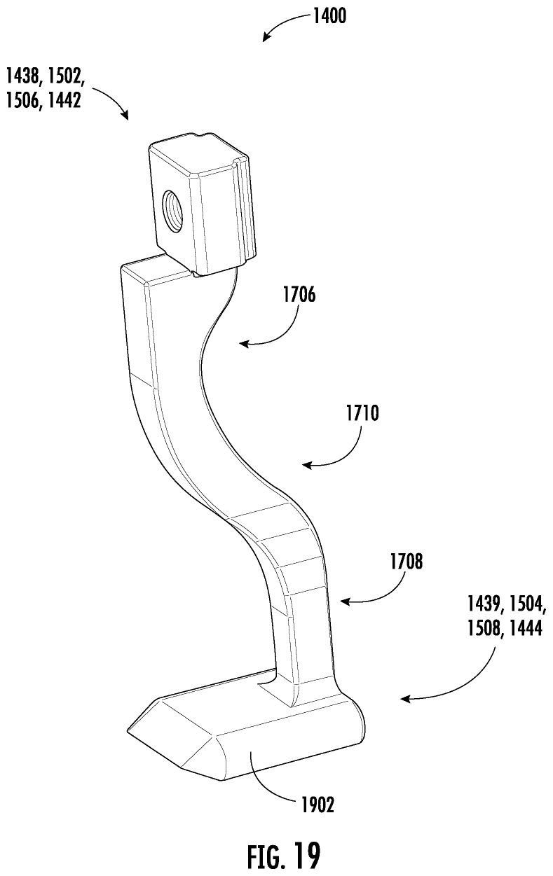

In some implementations, as shown in FIG. 19, the engagement feature can have a spike 1902 with a width greater than the first spike 1802 and the second spike 1804. For example, the spike 1902 can have at least one rectangular face, and can contact and engage with the bottom surface 1441. A width of the spike 1902 can span a width of the first spike 1802 and the second spike 1804 and a distance between the first spike 1802 and the second spike 1804. In some implementations, the spikes can be angled relative to the respective supporting member. In some implementations, at least one spike can extend from a center of the respective supporting member and in other implementations, the at least one spike is offset from the center.

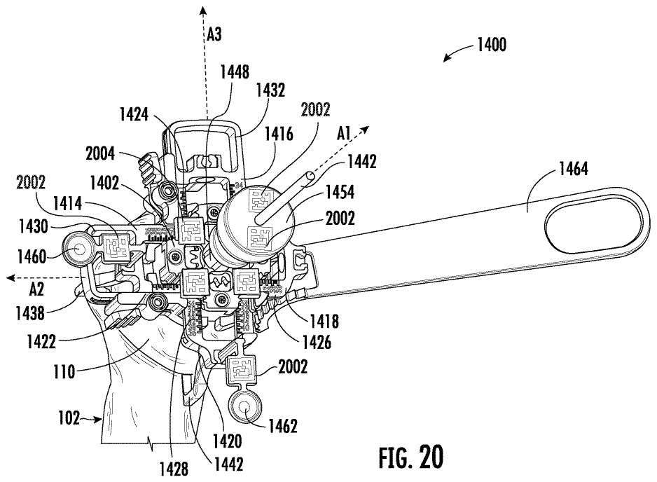

In some implementations, as shown in FIG. 20, the humeral head surgical guide 1400 can include one or more reference markers 2002 (e.g., tracking marker, etc.). The reference markers 2002 can be coupled to and disposed on various portions of the humeral head surgical guide 1400, such as but not limited to the first extender 1460, the second extender 1462, the guide body 1402, and the third handle 1454. The reference markers 2002 can include at least one of a barcode, quick response (QR) code, or other scannable identifier. The reference markers 2002 can be used or otherwise scanned by at least one sensor including at least one of a camera, augmented reality (AR), virtual reality (VR), or any other computing device, such as a mobile device or computer, to determine a relative position indicated by the reference markers 2002.

For example, the reference markers 2002 can include one or more base markers 2004 that may not move and can act as a reference point for other markers 2002. In some implementations, the base markers 2004 can be the reference markers 2002 coupled to and located on the guide body 1402. The reference markers 2002 can indicate an anterior-posterior distance between the base marker 2004 and reference markers 2002 that move perpendicular to the first axis A1. For example, the reference markers 2002 coupled to and located on the first extender 1460 (e.g., first reference marker) and the second extender 1462 (e.g., second reference marker) can be scanned and compared to the base markers 2004 to determine a distance of the reference markers 2002 disposed on the first extender 1460 and the second extender 1462 to the base markers on the guide body 1402. In some implementations, the distance between the reference marker 2002 disposed on the first extender 1460 and the base marker 2004 can correspond to at least one of the first distance or the third distance. In some implementations, the distance between the reference marker 2002 disposed on the second extender 1462 and the base marker 2004 can correspond to at least one of the first distance or the fourth distance. The reference marker 2002 disposed on the first extender 1460 can align with the first engagement feature 1439 and the reference marker 2002 disposed on the second extender 1462 can align with the fourth engagement feature 1444. The base marker 2004 can also indicate a distance between the base marker 2004 and the first axis A1 as such distance may not change. In some implementations, based on the distance between the reference markers 2002 disposed on the first extender 1460 and the second extender 1462 and the base markers 2004, the anatomical locations that the first extender 1460 and the second extender 1462 can be determined. Such as by comparing a known reference point (e.g., base markers 2004, guide body 1402), to an angle and distance from which the reference markers 2002 are located relative to the base markers 2004.

The reference markers 2002 can indicate superior-inferior distance between the base markers 2004 and reference markers 2002 that move along the first axis A1, such as the reference markers 2002 (e.g., third reference marker) disposed on the third handle 1454. For example, the reference markers 2002 disposed on the third handle 1454 can be scanned and compared to the base markers 2004 to determine the superior-inferior distance. In some implementations, the superior-inferior distance can be used to determine the fifth distance or the depth of the humeral head 110. In some implementations, the humeral head surgical guide 1400 can include reference markers 2002 for the guide body 1402, and for each movable member of the humeral head surgical guide 1400 such as but not limited to at least one of the first movable guide member 1414, the second movable guide member 1416, the third movable guide member 1418, the fourth movable guide member 1420, or the shaft 1448. The reference markers 2002 can be used to determine dimensions of the humeral head 110 and the center of the humeral head 110.

In implementations where the humeral head surgical guide 1400 includes reference markers 2002, instead of or in addition to determining respective distances using the markers and indicators, the surgeon can scan the reference markers 2002 to determine the distances and subsequently dimensions of the humeral head 110. For example, the surgeon can use at least one sensor including at least one of a mobile device, computer, camera, AR device (e.g., headset, glasses, etc.), VR device, or any other computing system to scan the reference markers 2002 and base markers 2004 to determine at least one of the first distance, the second distance, the third distance, the fourth distance, or the fifth distance. The computing system can output numerical values for at least one of the first distance, the second distance, the third distance, the fourth distance, or the fifth distance which can correspond to at least one of a width, length, or thickness of the humeral head 110. Using the numerical values, the surgeon or the computing system can determine a center of the humeral head 110 as well as select an implant corresponding to the dimensions of the humeral head 110. In some implementations, the computing system can be given the distances, and position the humeral head surgical guide 1400, such as in a virtual environment or in situations where the computing system is included in a surgical robot, in a clinical environment, on the humeral head 110 to set the center.

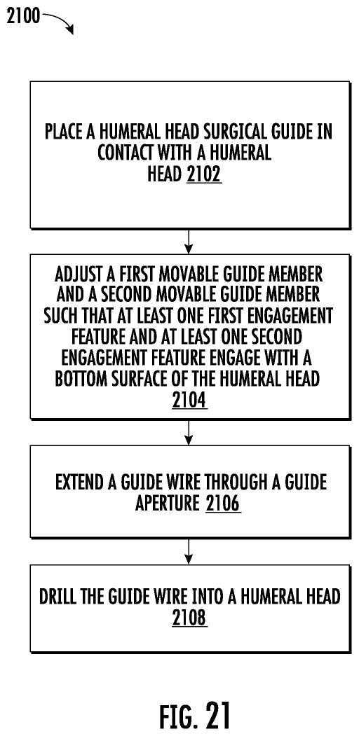

FIG. 21 depicts a flow diagram for an example method 2100 for using and centering a humeral head surgical guide (e.g., humeral head surgical guide 1400) on the humeral head 110. At block 2102, the method 2100 can include placing a humeral head surgical guide in contact with a humeral head (e.g., humeral head 110), the humeral head surgical guide including, a guide body including a first marker and a second marker, the guide body defining a guide aperture extending along a first axis at a center of the guide body; a first movable guide member coupled to the guide body including one or more first indicators, where the first movable guide member moves along a second axis perpendicular to the first axis; a first supporting member including at least one first engagement feature, the first supporting member coupled to and extending from the first movable guide member, where the first marker is oriented relative to the one or more first indicators to indicate a first distance between the first axis and the at least one first engagement feature; a second movable guide member coupled to the guide body including one or more second indicators, where the second movable guide member moves along a third axis perpendicular to the first axis and relative to the second axis; and a second supporting member including at least one second engagement feature, the second supporting member coupled to and extending from the second movable guide member, where the second marker is oriented relative to the one or more second indicators to indicate a second distance between the first axis and the at least one second engagement feature. For example, prior to the method 2100, skin and tissue surrounding and overlying the humeral head 110 may be moved or otherwise removed.

In some implementations, the first distance and the second distance can be used to determine dimensions of the humeral head to select an implant corresponding to the dimensions. In some implementations, the method 2100 can further include adjusting a shaft of the humeral head surgical guide including one or more fifth indicators such that the shaft contacts a top surface of the humeral head; and determining a depth of the humeral head based at least on the one or more fifth indicators indicating a fifth distance between the shaft and at least one of the at least one first engagement feature and the at least one second engagement feature. The fifth distance can be used to determine the depth of the humeral head, and the depth can be used to select the implant.

In some implementations, the humeral head surgical guide further includes a gear coupled with the first movable guide member and the second movable guide member, where the gear maintains the at least one first engagement feature and the at least one second engagement feature at a same distance from the first axis, where the gear causes the first movable guide member and the second movable guide member to be movable at a same rate.

At block 2104, the method 2100 can include adjusting the first movable guide member and the second movable guide member such that the at least one first engagement feature and the at least one second engagement feature engage with a bottom surface of the humeral head. To adjust the first movable guide member and the second movable guide member, a user, such as a surgeon or clinician, can apply a force, such as a pushing force, to at least one of the first movable guide member and the second movable guide member to move the at least one first engagement feature and the at least one second engagement feature to engage with the bottom surface of the humeral head. In some implementations, the first movable guide member and the second movable guide member can move together and in other implementations, first movable guide member and the second movable guide member can move independent of each other. Adjustment of at least the first movable guide member and the second movable guide member can facilitate determination of the center of the humeral head and secure the humeral head surgical guide on the humeral head.

In some implementations, the guide body can further include a third marker and a fourth marker, and the humeral head surgical guide can further include a third movable guide member coupled to the guide body including one or more third indicators, where the third movable guide member moves along the first axis; a third supporting member including at least one third engagement feature, the third supporting member coupled to and extending from the third movable guide member, where the third marker is oriented relative to the one or more third indicators to indicate a third distance between the first axis and the at least one third engagement feature; a fourth movable guide member coupled to the guide body including one or more fourth indicators, where the fourth movable guide member moves along the second axis; and a fourth supporting member including at least one fourth engagement feature, the fourth supporting member coupled to and extending from the fourth movable guide member, where the fourth marker is oriented relative to the one or more fourth indicators to indicate a fourth distance between the first axis and the at least one fourth engagement feature.

In such implementations, prior to drilling the guide wire, the method 2100 can further include adjusting the third movable guide member and the fourth movable guide member such that the at least one third engagement feature and the at least one fourth engagement feature engage with the bottom surface of the humeral head. In some implementations, the third movable guide member and the fourth movable guide member can be adjusted via at least one of the first movable guide member or the second movable guide member. The method 2100 can further include determining one or more dimensions of the humeral head based at least on the first distance, the second distance, the third distance, and the fourth distance. The dimensions (e.g., at least width and length) can be used to select an implant suitable for the respective humeral head. In some implementations, the method 2100 can include selecting a shoulder implant based at least on the one or more dimensions.

At block 2106, the method 2100 can include extending a guide wire through the guide aperture. Once the first movable guide member and the second movable guide member are adjusted such that the at least one first engagement feature and the at least one second engagement feature engage with a bottom surface of the humeral head, the guide aperture can be aligned with the center of the humeral head, and the guide wire can be extended through the guide aperture.

At block 2108, the method 2100 can include drilling the guide wire into the humeral head. To attach the guide wire to the humeral head, the guide wire can be attached to a drill while extending through the guide aperture, and drilled into the center of the humeral head. In some implementations, the method 2100 can include implanting the shoulder implant. In some implementations, implantation can follow preparation of the humerus and removal of the guide wire. In some implementations, adjustment of at least the first movable guide member and the second movable guide member determines a center of the humeral head, and the guide wire is drilled into the center of the humeral head.

For example, a surgeon can use the humeral head surgical guide 1400 during a shoulder arthroplasty procedure to center a guide wire 1446 in the humeral head 110 and to obtain dimensional measurements for implant selection. Following exposure of the humeral head 110 (e.g., incisions along deltopectoral, anterolateral, posterior, etc.) and removal of obstructing soft tissues and osteophytes, the surgeon can position the guide body 1402 of the humeral head surgical guide on or adjacent to the superior (e.g., top) surface of the humeral head 110 such that the guide aperture 1702 and the corresponding first axis A1 are approximately aligned with the visual center of the humeral head 110. The guide body 1402 can be oriented so that the second and third axes A2, A3 of motion of the movable guide members (e.g., first movable guide member 1414, second movable guide member 1416, third movable guide member 1418, fourth movable guide member 1420) are suitably aligned relative to the humeral head 110, for example roughly in anteroposterior and mediolateral directions, while ensuring that the supporting members (e.g., first supporting member 1438, second supporting member 1502, third supporting member 1506, fourth supporting member 1442) and engagement features (e.g., first engagement feature 1439, second engagement feature 1504, third engagement feature 1508, fourth engagement feature 1444) can extend around and under the humeral head 110 to contact the inferior or bottom surface 1441 of the humeral head 110 and define an anatomical neck of the humeral head 110.

Once the guide body 1402 is placed, the surgeon can adjust the first movable guide member 1414 and the second movable guide member 1416 to position the first engagement feature 1439 and the second engagement feature 1504 against the bottom surface 1441 of the humeral head 110. In some implementations, this adjustment is performed by applying a pushing force to the first movable guide member 1414, the second movable guide member 1416, or both, thereby advancing the respective supporting members 1438, 1502 until the first and second engagement features 1439, 1504 are brought into firm contact with opposing regions of the inferior surface 1441 of the humeral head 110. Where a gear 1458 is provided to couple the first and second movable guide members 1414, 1416, advancement of one guide member can result in symmetric movement of both guide members at the same rate such that the first and second engagement features 1439, 1504 move toward and engage the humeral head 110 in a coordinated and equidistant manner relative to the first axis A1. In implementations without a gear 1458, the first movable guide member 1414 and the second movable guide member 1416 can be adjusted independently with the surgeon iteratively advancing each until both engagement features 1439, 1504 are in secure contact with the bottom surface 1441, therefore stabilizing and centering the guide body 1402 on the humeral head 110.

With the first and second movable guide members 1414, 1416 positioned, the surgeon can observe the relative positions of the first marker 1406 and the one or more first indicators 1422 to determine the first distance between the first axis A1 and the first engagement feature 1439, and can similarly observe the relative positions of the second marker 1408 and the one or more second indicators 1424 to determine the second distance between the first axis A1 and the second engagement feature 1504. These distances can be used to determine one or more dimensions of the humeral head 110, for example diameters or radii in orthogonal directions, which may be applied in selecting an appropriately sized humeral head implant. In implementations where the humeral head surgical guide 1400 further includes the third marker 1410, the fourth marker 1412, the third movable guide member 1418, and the fourth movable guide member 1420, the surgeon can adjust the third movable guide member 1418 along the second axis A2 to bring the third engagement feature 1508 into contact with the bottom surface 1441 of the humeral head 110, and can adjust the fourth movable guide member 1420 along the second axis A2 to bring the fourth engagement feature 1444 into contact with the bottom surface 1441 of the humeral head 110. These adjustments can be achieved by directly manipulating the third and fourth movable guide members 1418, 1420 and/or indirectly via coordinated motion with the first and second movable guide members 1414, 1416, where provided.

Once the at least one third engagement feature 1508 and the at least one fourth engagement feature 1444 engage the bottom surface 1441 of the humeral head 110, the surgeon can observe the relative positions of the third marker 1410 and the one or more third indicators 1426 to determine the third distance between the first axis A1 and the third engagement feature 1508, and observe the relative positions of the fourth marker 1412 and the one or more fourth indicators 1428 to determine the fourth distance between the first axis A1 and the fourth engagement feature 1444. The first distance, the second distance, the third distance, and the fourth distance collectively can define multiple dimensions of the humeral head 110, such as width, length, or ellipticity in different planes, which can be compared to known implant geometries to select a shoulder implant corresponding to the measured dimensions. In some implementations, additional stability is provided by four-point engagement of the humeral head 110 via the first, second, third, and fourth engagement features 1439, 1504, 1508, 1444, which can further ensure accurate centering of the guide aperture 1702 relative to the humeral head center.

In some implementations, the first movable guide member 1414 and the third movable guide member 1418 are adjusted such that respective engagement features contact and engage with the humeral head 110. Following adjustment along the second axis A2, the second movable guide member 1416 and the fourth movable guide member 1420 are adjusted such that respective engagement features contact and engage with the humeral head 110. In various embodiments, the first movable guide member 1414 and the third movable guide member 1418 are further adjusted after adjustment of the second movable guide member 1416 and the fourth movable guide member 1420.

In some implementations, the humeral head surgical guide 1400 can further include a shaft 1448 extending generally along the first axis A1 and bearing one or more fifth indicators 1450 for depth measurement. With the guide body 1402 stabilized and the inferior engagement features 1439, 1504, 1508, 1444 in contact with the bottom surface 1441 of the humeral head 110, the surgeon can advance the shaft 1448 toward the superior surface of the humeral head 110 until the end 1704 of the shaft 1448 contacts the top surface of the humeral head 110. The position of the shaft 1448 relative to the fifth indicators 1450 can then be used to determine a fifth distance between the shaft 1448 and at least one of the first engagement feature 1439 and the second engagement feature 1504 (or in some embodiments between the shaft 1448 and any selected engagement feature 1508, 1444), therefore providing a measurement corresponding to the depth or height of the humeral head 110. This depth measurement can be used alone or in combination with the first, second, third, and fourth distances to further characterize the humeral head size and shape and to refine selection of the shoulder implant.

After the humeral head surgical guide 1400 has been adjusted and the humeral head dimensions have been determined as described, the surgeon can use the guide aperture 1702 to place a guide wire 1446 into the center of the humeral head 110. With the guide body 1402 maintained at a centered, stabilized position on the humeral head 110, the surgeon can extend the guide wire 1446 through the guide aperture 1702 along the first axis A1 until the distal tip of the guide wire 1446 contacts the superior surface of the humeral head 110 at a location corresponding to the geometric center determined by the engagement features 1439, 1504, 1508, 1444. The guide wire 1446 can then be coupled to a drill while still extending through the guide aperture 1702, and the surgeon can drill the guide wire 1446 into the humeral head 110 along the first axis A1 to a desired depth. Because the engagement features 1439, 1504, 1508, 1444 maintain the humeral head surgical guide 1400 in a centered and secure orientation, the guide wire 1446 is drilled substantially into the center of the humeral head 110. Once the guide wire 1446 is implanted, the surgeon can withdraw the humeral head surgical guide 1400 by retracting or releasing the movable guide members 1414, 1416, 1418, 1420 to disengage the engagement features 1439, 1504, 1508, 1444 from the humeral head 110 and lifting the guide body 1402 away, leaving the guide wire 1446 in place. In some implementations, to release the movable guide members 1414, 1416, 1418, 1420, the surgeon actuates the first actuator 1434 and the second actuator 1436 to move the humeral head surgical guide 1400 to a position prior to adjustment such that the engagement features 1439, 1504, 1508, 1444 no longer contact the humeral head 110. The centrally placed guide wire 1446 can then serve as a reference for subsequent preparation of the humerus 102, including reaming, cutting, or other bone preparation steps, and for the implantation of the selected shoulder implant following removal of the guide wire 1446.

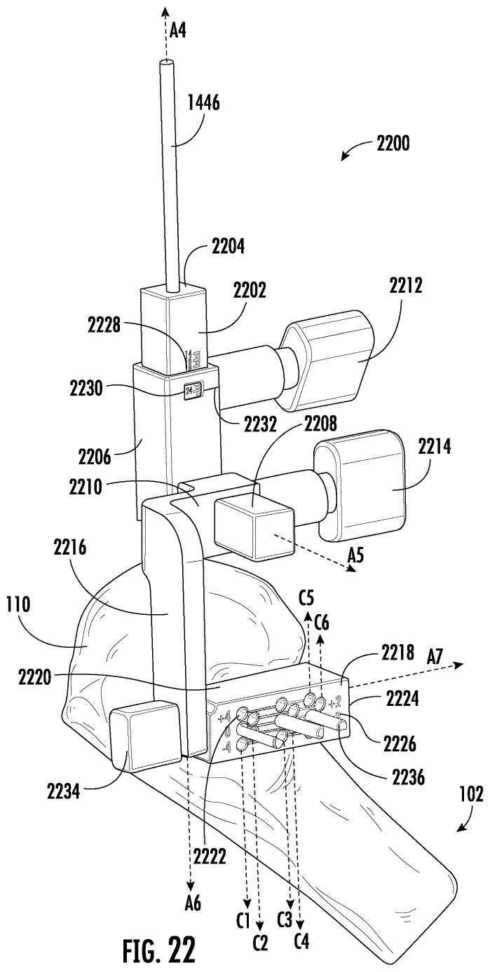

Referring now to FIGS. 22-25, FIGS. 22-25 depict an example humeral head cutting apparatus 2200 (e.g., first surgical apparatus, etc.). The humeral head cutting apparatus 2200 can be an example of the cutting jig 500 or 600. The humeral head cutting apparatus 2200 can be configured to cut the humeral head 110 to prepare the humerus 102 for shoulder implant implantation, such as by resecting the humeral head 110 and forming a planar surface. The humeral head cutting apparatus 2200 can be used following attachment of the guide wire 1446 to the center of the humeral head 110. For example, the humeral head cutting apparatus 2200 can be used following centering of the humeral head 110 using the humeral head surgical guide 1400 and attaching of the guide wire 1446 to the center of the humeral head 110. The humeral head cutting apparatus 2200 can be attached to the guide wire 1446 such that the guide wire 1446 extends through the humeral head cutting apparatus 2200 to position the humeral head cutting apparatus 2200 for cutting.

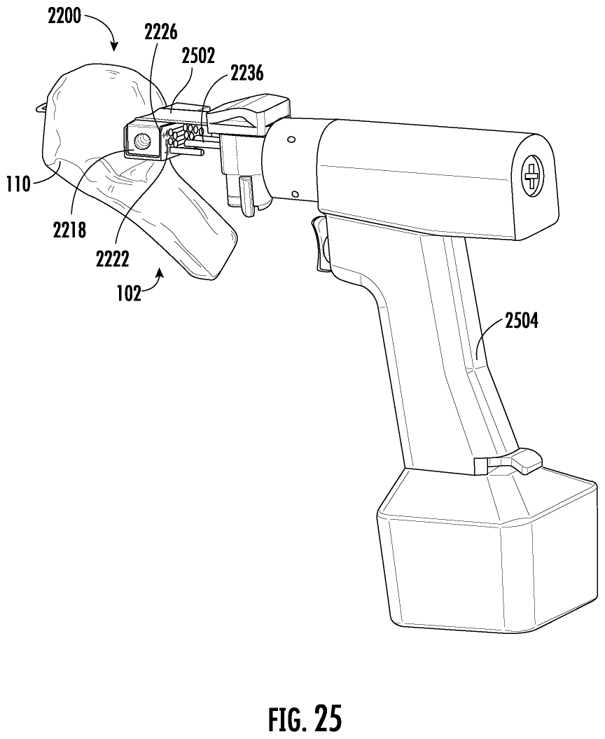

The humeral head cutting apparatus 2200 can include at least one first body 2202. The first body 2202 can define at least one first aperture 2204. In some implementations, the first body 2202 has a rectangular shape and in other implementations, the first body 2202 has a cylindrical or any other shape. The first body 2202 and the first aperture 2204 can extend along a fourth axis A4. The humeral head cutting apparatus 2200 can be attached to the guide wire 1446 via the first aperture 2204. For example, as shown in FIG. 25, the first aperture 2204 can receive the guide wire 1446 while the guide wire 1446 is attached to the center of the humeral head 110, and the guide wire 1446 can extend through the first aperture 2204. While extending through the first aperture 2204, the guide wire 1446 can extend along the fourth axis A4. In some implementations, the first body 2202 and the first aperture 2204 are centered on the fourth axis A4. The first body 2202 can have a length between 40 to 80 mm, inclusive, and can have a width between 5 to 20 mm, inclusive. In some implementations, the first body 2202 has a length less than 40 or greater than 80 mm, and a width less than 5 or greater than 20 mm.

The humeral head cutting apparatus 2200 can include at least one first movable member 2206 (e.g., sliding member, carriage, etc.). The first movable member 2206 can be an example of the arm 602. The first movable member 2206 can extend around the first body 2202. In some implementations, the first movable member 2206 is removably coupled to the first body 2202. The first movable member 2206 can be movable along the first body 2202. For example, the first movable member 2206 can move along the fourth axis A4. The first movable member 2206 can extend along the fourth axis A4. In some implementations, the first movable member 2206 is centered on the fourth axis A4. The first movable member 2206 can have a length between 20 to 60 mm, inclusive, and a width between 8 to 25 mm, inclusive. In some implementations, the first movable member 2206 can have a length less than 20 or greater than 60 mm, and a width less than 8 or greater than 25 mm. The first movable member 2206 can have a length less than the first body 2202, and a width greater than the first body 2202 such that the first movable member 2206 can extend around the first body 2202. In some implementations, the first movable member 2206 has a length equal to or greater than the first body 2202.

The humeral head cutting apparatus 2200 can include at least one second body 2208 coupled to the first movable member 2206. In some implementations, the second body 2208 is integral with the first movable member 2206 such that the first movable member 2206 and the second body 2208 form a monolithic structure. In some implementations, the second body 2208 is contiguous with the first movable member 2206. The second body 2208 can extend along a fifth axis A5. The fifth axis A5 can be perpendicular to the fourth axis A4. In some implementations, the fifth axis A5 is at an angle relative to the fourth axis A4 other than 90 degrees, such as less than or greater than 90 degrees. In some implementations, the second body 2208 can be centered on the fifth axis A5. In some implementations, the second body 2208 has a length of between 15 to 50 mm, inclusive, and a width of between 3 to 20 mm, inclusive. In some implementations, the second body 2208 has a length of less than 15 or greater than 50 mm, and a width of less than 3 or greater than 20 mm. In some implementations, the second body 2208 has a length less than the first body 2202 and the first movable member 2206, and a width less than the first movable member 2206. In some implementations, a length of the second body 2208 is greater than or equal to at least one of the first body 2202 or the first movable member 2206, and a width greater than or equal to the first movable member 2206.

The humeral head cutting apparatus 2200 can include at least one second movable member 2210. The second movable member 2210 can be an example of arm 608. The second movable member 2210 can extend around the second body 2208. In some implementations, the second movable member 2210 is removably coupled to the second body 2208. The second movable member 2210 can be movable along the second body 2208. For example, the second movable member 2210 can move along the fifth axis A5. The second movable member 2210 can extend along the fifth axis A5. In some implementations, the second movable member 2210 is centered on the fifth axis A5. The second movable member 2210 can have a length between 3 to 25 mm, inclusive, and a width between 5 to 30 mm, inclusive. In some implementations, the second movable member 2210 can have a length less than 3 or greater than 25 mm, and a width less than 5 and greater than 30 mm. The length of the second movable member 2210 can be less than the length of the second body 2208, and a width of the second movable member 2210 can be greater than the second body 2208. In some implementations, the length of the second movable member 2210 is equal to or greater than the length of the second body 2208.

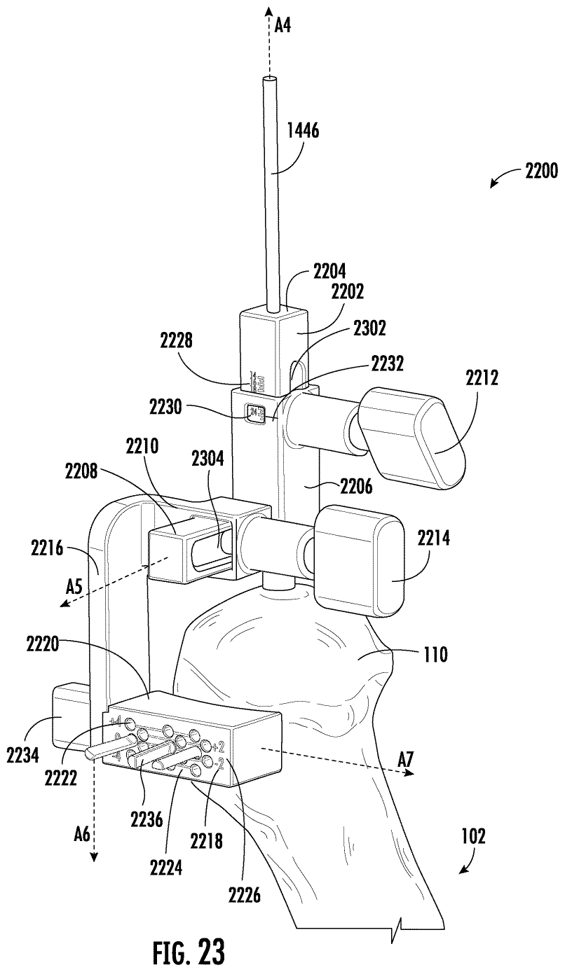

The humeral head cutting apparatus 2200 can include at least one first fastener 2212. The first fastener 2212 can be an example of knob 604. The first fastener 2212 can be coupled to the first movable member 2206. In some implementations, the first fastener 2212 can be in contact with and extend through the first movable member 2206 to contact the first body 2202. In some implementations, as shown in FIG. 23, the first body 2202 can define at least one first receiving space 2302. The first fastener 2212 can be coupled to and extend through the first movable member 2206 to contact and engage with the first receiving space 2302. The first fastener 2212 can allow and stop movement of the first movable member 2206 along the first body 2202.

For example, while the first fastener 2212 is in contact with the first body 2202 at the first receiving space 2302, the first movable member 2206 may be locked in a position. To move the first movable member 2206, the first fastener 2212 can be actuated, such as rotated, such that the first fastener 2212 is removed from contact with the first body 2202, allowing the first movable member 2206 to move along the first body 2202. In some implementations, the first receiving space 2302 can define a range of motion of the first movable member 2206. For example, the first movable member 2206 may not move beyond a length of the first receiving space 2302. The first fastener 2212 can include at least one of a knob, lever, dial, or any other device to allow and stop movement of the first movable member 2206.

The humeral head cutting apparatus 2200 can include at least one second fastener 2214. The second fastener 2214 can be an example of knob 612. The second fastener 2214 can be coupled to the second movable member 2210. In some implementations, the second fastener 2214 can be in contact with and extend through the second movable member 2210 to contact the second body 2208. In some implementations, as shown in FIG. 23, the second body 2208 can define at least one second receiving space 2304. The second fastener 2214 can be coupled to and extend through the second movable member 2210 to contact and engage with the second receiving space 2304. The second fastener 2214 can allow and stop movement of the second movable member 2210 along the second body 2208.

For example, while the second fastener 2214 is in contact with the second body 2208 at the second receiving space 2304, the second movable member 2210 may be locked in a position. To move the second movable member 2210, the second fastener 2214 can be actuated, such as rotated, such that the second fastener 2214 is removed from contact with the second body 2208, allowing the second movable member 2210 to move along the second body 2208. In some implementations, the second receiving space 2304 can define a range of motion of the second movable member 2210. For example, the second movable member 2210 may not move beyond a length of the second receiving space 2304. The second fastener 2214 can include at least one of a knob, lever, dial, or any other device to allow and stop movement of the second movable member 2210.

The humeral head cutting apparatus 2200 can include at least one arm 2216. The arm 2216 can be coupled to the second movable member 2210. In some implementations, the second movable member 2210 includes the arm 2216 such that the arm 2216 is integrated with or a monolithic structure with the second movable member 2210. In some implementations, the arm 2216 is contiguous with the second movable member 2210. The arm 2216 can partially extend along a sixth axis A6 parallel to and offset from the fourth axis A4. In some implementations, the sixth axis A6 is perpendicular to the fifth axis A5 and in other implementations, the sixth axis A6 is angled relative to the fifth axis A5 such as at an angle less than or greater than 90 degrees. In some implementations, the sixth axis A6 is angled relative to the fourth axis A4. The arm 2216 can move along with movement of the second movable member 2210. The arm 2216 can include a curved portion that allows the arm 2216 to partially extend along the sixth axis A6, and partially extend along an axis perpendicular to the fifth axis A5 and the sixth axis A6.

In some implementations, a total length of the arm 2216 extending from the second movable member 2210 and along the sixth axis A6 can be between 60 to 110 mm, inclusive. In some implementations, the length of the arm 2216 can be less than 60 or greater than 110 mm. The width of the arm 2216 along the axis perpendicular to the fifth axis and the sixth axis A6 can be between 2 to 10 mm, inclusive. In some implementations, the width of the arm 2216 can be less than 2 or greater than 10 mm.

The humeral head cutting apparatus 2200 can include at least one cutting support member 2218. The cutting support member 2218 can be removably coupled with the arm 2216. In some implementations, the cutting support member 2218 is coupled with or otherwise integrated with the arm 2216. The cutting support member 2218 can extend along a seventh axis A7. In some implementations, the seventh axis A7 is perpendicular to the fifth axis A5, the sixth axis A6, and the fourth axis A4. In some implementations, the seventh axis A7 is angled relative to the sixth axis A6, such as at an angle less than or greater than 90 degrees. In some implementations, the cutting support member 2218 can have a rectangular shape or any other shape. In some implementations, the cutting support member 2218 can have a length of between 20 to 55 mm, inclusive, and a width of between 10 to 30 mm, inclusive. In some implementations, the length of the cutting support member 2218 is less than 20 or greater than 50 mm, and the width is less than 10 or greater than 30 mm. In some implementations, a height of the cutting support member 2218 is between 5 to 30 mm, inclusive, and in other implementations, the height of the cutting support member 2218 is less than 5 or greater than 30 mm.

As shown in FIG. 25, the cutting support member 2218 can support and guide a cutting member 2502 (e.g., saw blade, etc.). The cutting member 2502 can be used to perform osteotomy. The cutting member 2502 can be an example of the saw blade 518. The cutting member 2502 can be a saw blade, blade, knife, or other apparatus capable of cutting into at least one of the humeral head 110 or the humerus 102. The cutting member 2502 can be attached to a saw 2504 or other power source that can cause the cutting member 2502 to oscillate along, above, below, or proximate to the cutting support member 2218 to cut into the at least one of the humeral head 110 or the humerus 102 to form the planar surface. For example, the cutting support member 2218 can include a top surface 2220 (e.g., guiding surface, cutting surface, etc.) that guides the cutting member 2502. The cutting member 2502, prior to oscillating, can be placed in contact with the top surface 2220, and the cutting member 2502 can remain in contact with the top surface 2220 while oscillating to guide the cutting member 2502. In some implementations, the cutting member 2502 may not be in contact with the top surface 2220 while oscillating, and the top surface 2220 can be used as a reference point by, for example, a surgeon or surgical robot. In some implementations, the top surface 2220 can determine a cutting plane of the cutting member 2502 (e.g., plane at which the cutting member 2502 resects the humeral head 110).

To secure a position of the cutting support member 2218, the cutting support member 2218 can define a plurality of cutting support apertures 2222. The plurality of cutting support apertures 2222 can herein be referred to as apertures 2222. The apertures 2222 can be an example of the holes 512 or 624 and holes 514 or 626. Each of the cutting support apertures 2222 can be located at different positions of the cutting support member 2218. For example, the cutting support member 2218 can include a first surface 2224 contiguous with the top surface 2220 and a second surface 2402 opposite the first surface 2224 and contiguous with the top surface 2220. The cutting support apertures 2222 can extend from the first surface 2224 to the second surface 2402. In some implementations, the second surface 2402 can be aligned with the humeral head 110 and in some implementations, at least partially contact the humeral head 110. In some implementations, the second surface 2402 can be contoured to match or mimic a contour of the humeral head 110 such that the second surface 2402 is at least partially curved.

The cutting support apertures 2222 can include sets of cutting support apertures 2222 located along different cutting axes. For example, a first set of cutting support apertures 2222 are located along a first cutting axis C1, a second set of cutting support apertures 2222 are located along a second cutting axis C2, a third set of cutting support apertures 2222 are located along a third cutting axis C3, a fourth set of cutting support apertures 2222 are located along a fourth cutting axis C4, a fifth set of cutting support apertures 2222 are located along a fifth cutting axis C5, and a sixth set of cutting support apertures 2222 are located along a sixth cutting axis C6. The cutting axes C1 and C2 can be closer to each other than the other axes (e.g., axes C3, C4, C5, C6). The cutting axes C3 and C4 can be closer to each other than the other axes. The cutting axes C5 and C6 can be closer to each other than the other axes. The cutting axes (e.g., axes C1, C2, C3, C4, C5, C6) can be parallel and offset from each other. The subsets of apertures 2222 along axes C1, C3, and C5 can include three apertures 2222 while the subsets of apertures 2222 along axes C2, C4, and C6. The apertures 2222 along axes C2, C4, and C6 can be offset from the apertures 2222 along C1, C3, and C5. The first, second, and third apertures 2222 along axes C1, C3, and C5 can be aligned with each other, respectively. The first and second apertures 2222 along axes C2, C4, and C6 can be aligned with each other, respectively.

The cutting support member 2218 can include a plurality of cutting indicators 2226 aligned with the cutting support apertures 2222. For example, each of the cutting indicators 2226 can be aligned with a set of first apertures 2222 along the axes C1, C3, and C5, a set of second apertures 2222 along the axes C1, C3, and C5, a set of third apertures 2222 along the axes C1, C3, and C5, a set of first apertures 2222 along the axes C2, C4, and C6, and a set of second apertures 2222 along the axes C2, C4, and C6. Consequently, the set of apertures 2222 along axis C2 can be offset from the set of apertures 2222 along axis C1, and so on.

The cutting indicators 2226 can indicate different securement positions for the cutting support member 2218. For example, one of the cutting indicators 2226 can indicate “0” which can correspond to the position of the first movable member 2206 along the first body 2202. The one of the cutting indicators 2226 indicating “0” can correspond to the set of second apertures 2222 along the axes C1, C3, and C5, and the cutting support member 2218 can be secured at a subset of the cutting apertures 2222 corresponding to the cutting indicators 2226, as described herein.

In some implementations, the first body 2202 includes a plurality of body indicators 2228. The body indicators 2228 can be located along a length of the first body 2202 and can be located on a surface different from the first receiving space 2302. The first movable member 2206 can define a movable member aperture 2230 (e.g., window, etc.) which can be aligned with at least one of the body indicators 2228. For example, the movable member aperture 2230 can extend through the first movable member 2206 to display a subset of the body indicators 2228. The first movable member 2206 can further include a movable member marker 2232 to align with at least one of the body indicators 2228. The marker 2232 can be a line extending across an outer surface of the first movable member 2206, and can indicate a position of the first movable member 2206 and the second movable member 2210.

The marker 2232 along with the body indicators 2228 and the first fastener 2212 can be used to set the humeral head cutting apparatus 2200 to a position. In some implementations, the position corresponds to a cutting plane of the humeral head cutting apparatus 2200, such as the top surface 2220. In some implementations, the position can correspond to the depth of the humeral head 110 determined by the humeral head surgical guide 1400. For example, the body indicators 2228 can correspond to and include values of the fifth indicators 1450. Based on the fifth distance, the first fastener 2212 can release and allow movement of the first movable member 2206 along the first body 2202. The first movable member 2206 can be adjusted such that the marker 2232 aligns with a body indicator 2228 corresponding to the fifth distance and the depth. In some implementations, the first movable member 2206 is adjusted to a position different from the fifth distance, such that the cutting plane may not directly correspond to the depth of the humeral head 110. Once the marker 2232 and the body indicator 2228 are aligned, the first fastener 2212 can be adjusted such that the first fastener 2212 contacts and engages with the first receiving space 2302 to lock a position of the first movable member 2206. Movement of the first movable member 2206 can also move the second movable member 2210 and subsequently the cutting support member 2218 along the fourth axis A4.

In some implementations, a user can adjust the humeral head cutting apparatus 2200 to determine and set the cutting plane. For example, the user can adjust a position of the cutting support member 2218 using at least one of the first fastener 2212 and the first movable member 2206 or the second fastener 2214 and the second movable member 2210 to select a resection (e.g., cutting) plane (e.g., level) as determined and indicated by at least the cutting support member 2218. For example, a position of the first movable member 2206 along the first body 2202 as set and adjustable by the first fastener 2212 can determine a depth or height (e.g., vertical position) at which the humeral head 110 is cut, and a position of the second movable member 2210 along the second body 2208 as set and adjustable by the second fastener 2214 can determine a lateral position of the cutting support member 2218 which in some implementations, can correspond to the width or length of the humeral head 110.

In some implementations, to allow the user to set an angle at which the humeral head 110 is cut, the humeral head cutting apparatus 2200 can include a third fastener 2234. The third fastener 2234 can be coupled to and extend through the arm 2216 and can contact and engage with the cutting support member 2218. In some implementations, the third fastener 2234 can allow for angular adjustment of the cutting support member 2218. For example, in response to being released, the third fastener 2234 can allow for angular adjustment or rotation of the cutting support member 2218. In response to being tightened or otherwise actuated, the third fastener 2234 can lock an angular position of the cutting support member 2218. Thus, the user can also set an angle at which the humeral head 110 is cut using at least the third fastener 2234. In some implementations, the third fastener 2234 allows for removable coupling of the cutting support member 2218 with the arm 2216. In some implementations, the third fastener 2234 includes a knob, dial, lever, or any other device.

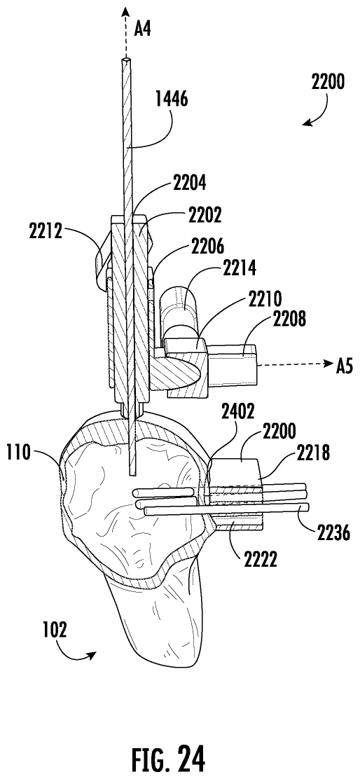

The humeral head cutting apparatus 2200 can include a plurality of pins 2236 (e.g., olive pins, etc.). The pins 2236 can be an example of the olive pins 516 or 628. The pins 2236 can extend through a subset of the cutting support apertures 2222 to secure the cutting support member 2218 to the humeral head 110. In response to setting at least one of the vertical, lateral, or angular position of the cutting support member 2218, a user can insert the pins 2236 into the subset of apertures 2222 using, for example, a hammer or any other tool to drive the pins 2236 through the apertures 2222 and into the humeral head 110, as shown in at least FIG. 24, to secure the cutting support member 2218 to the humeral head 110. The various positions of the apertures 2222 can allow for options for the user to select to secure the cutting support member 2218 to the humeral head 110. For example, the user can determine quality at different portions of the humeral head 110 aligned with different apertures 2222, geometry of the humeral head 110, or any other factors to select the subset of the apertures 2222 for which to drive the pins 2236 into. The apertures 2222 thus allow for flexibility to attach the pins 2236 and the cutting support member 2218 to avoid weak or damaged bone regions and for increased stability of the cutting support member 2218. The apertures 2222 aligned with the indicator 2226 of “0” can be a reference point, and the user can select a subset of the apertures 2222 corresponding to indicators 2226 of positive 2, negative 2, positive 4, or negative 4 from the indicator 2226 indicating “0.” In addition, the various positions of the apertures 2222 allow the surgeon to increase humeral head 110 cuts (e.g., depth, etc.) by, for example but not limited to, 2 millimeters (mm) per row of apertures 2222/

In some implementations, selection of the subset of the apertures 2222 can indicate a distance of the cutting support member 2218, such as the second surface 2402, from the humeral head 110. For example, the apertures 2222 aligned with indicator 2226 “0” can serve as a reference point, and in response to the pins 2236 extending through the apertures 2222 aligned with indicator 2226 “0”, the cutting member 2502 can cut the humeral head 110 at a first depth. In response to the apertures 2222 aligned with indicator, for example, “+2,” the cutting depth can change, such as by 2 millimeters (mm). For example, the reference point can correspond to a distance of 10 mm from the humeral head 110, and the apertures 2222 aligned with indicator 2226 “+2” can correspond to a distance of 12 mm from the humeral head 110. The apertures 2222 aligned with indicator 2226 “+2” can also be offset from the reference point which can also adjust the distance between the humeral head 110 and the second surface 2402. Consequently, the pins 2236 extending through “−2” or “−4” can reduce the distance while the pins 2236 extending through “+4” can further increase the distance of the second surface 2402 from the humeral head 110, thereby changing the cutting depth and allowing the user to configure and set a depth at which the humeral head 110 is cut. For example, as the cutting depth increases, an amount of the humeral head 110 resected can increase along the axis A5, and thus the user can determine the cutting depth based on an amount of the humeral head 110 desired to be resected. In some implementations, selection of the apertures 2222 to drive the pins 2236 through can adjust a position of the top surface 2220.

Following securement of the pins 2236 through the subset of apertures 2222 and to the humeral head 110, as shown in FIG. 25, the guide wire 1446 and the first body 2202 along with the second body 2208 and the arm 2216 (e.g., portion of the humeral head cutting apparatus 2200) can be removed from the guide wire 1446. Consequently, prior to cutting the humeral head 110, only the cutting support member 2218 is coupled to the humeral head 110 via the pins 2236. The portion of the humeral head cutting apparatus 2200 can be decoupled from the cutting support member 2218 via the third fastener 2234. Following removal of the guide wire 1446 and the portion of the humeral head cutting apparatus 2200, the cutting member 2502 cuts the humeral head 110 along the top surface 2220. Following the cutting, the pins 2236 can be removed from the humeral head 110, and the humeral head cutting apparatus 2200 can be removed from the guide wire 1446. Cutting of the humeral head 110 can allow at least one surface of the humeral head 110 to be planar, and may include removing a portion of the humeral head 110.

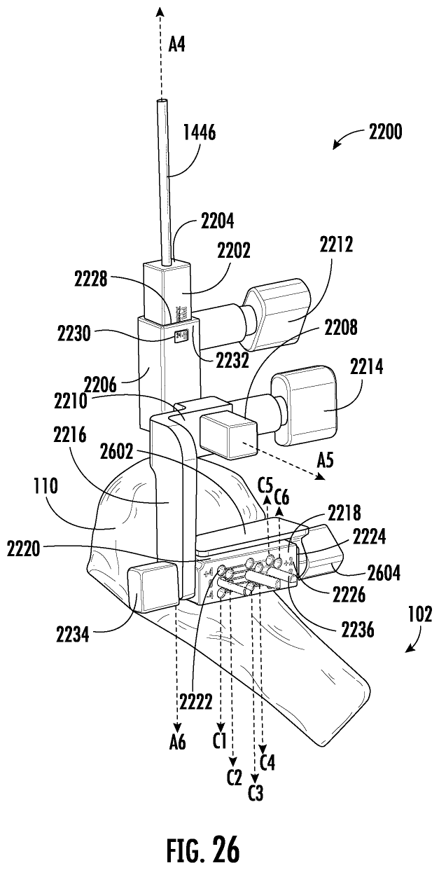

In some implementations, as shown in FIG. 26, the humeral head cutting apparatus 2200 can include at least one platform 2602 (e.g., shelf, plate, etc.). The platform 2602 can be removably coupled with the cutting support member 2218 and at least partially face the top surface 2220. In some implementations, the platform 2602 can be located above the top surface 2220 such that the cutting member 2502 can extend between the top surface 2220 and the platform 2602. In some implementations, the platform 2602 can provide additional guidance along with the top surface 2220 for the cutting member 2502 to cut the humeral head 110. In some implementations, the cutting member 2502 can be in contact with and oscillate on the platform 2602. In some implementations, at least one of the top surface 2220 or the platform 2602 constrain movement of the cutting member 2502 such that the cutting member 2502 cuts at the desired height, position and angle set by the humeral head cutting apparatus 2200.

In some implementations, the humeral head cutting apparatus 2200, as shown in FIG. 26, can include a fourth fastener 2604. The fourth fastener 2604 can be coupled with at least one of the platform 2602 or the cutting support member 2218, and can removably couple the platform 2602 to the cutting support member 2218. For example, the platform 2602 can be coupled to and removed from the cutting support member 2218 via the fourth fastener 2604.

In some implementations, the cutting support member 2218 defines at least one slot. For example, the slot can extend between or include the cutting support apertures 2222. In some implementations, the cutting support member 2218 defines one slot per indicator 2226. In such implementations, the cutting member 2502 can extend into a slot to cut the humeral head 110. For example, the cutting member 2502 can contact the cutting support member 2218 within the slot, and oscillate within the slot to cut the humeral head 110.

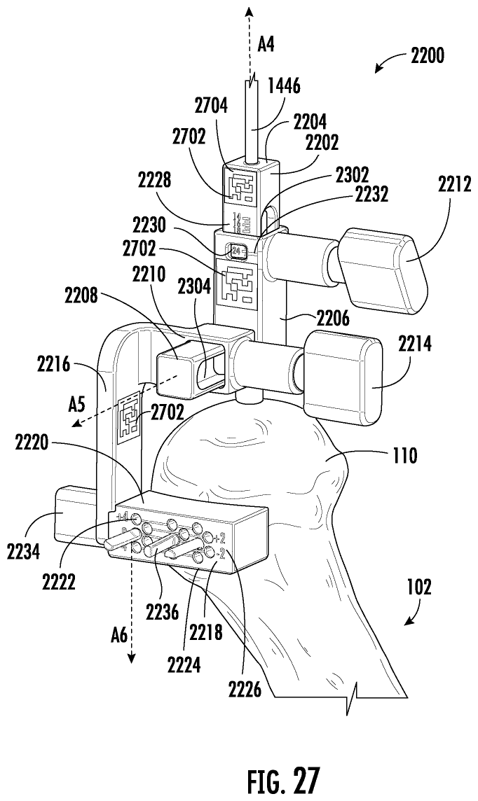

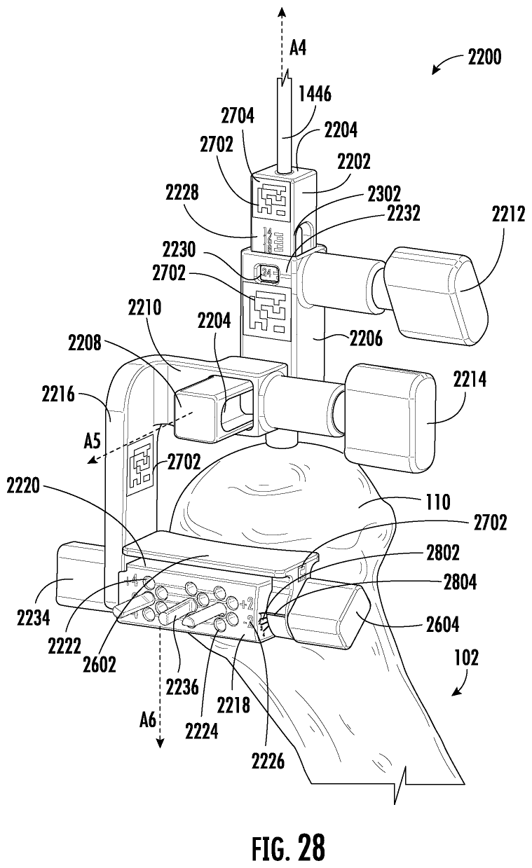

In some implementations, as shown in FIGS. 27-28, the humeral head cutting apparatus 2200 can include reference markers 2702, which can be similar to the reference markers 2002. The reference markers 2702 can be coupled to, for example but not limited to, the first body 2202, the first movable member 2206, or the arm 2216. The reference markers 2702 can include at least one of a barcode, quick response (QR) code, or other scannable identifier. The reference markers 2702 can be used or otherwise scanned by at least one of augmented reality (AR), virtual reality (VR), or any other computing device, such as a mobile device or computer, to determine a relative position indicated by the reference markers 2702.

For example, the reference markers 2702 can include one or more base markers 2704 that may not move and can act as a reference point for other markers 2702. In some implementations, the base markers 2704 can be the reference markers 2702 coupled to and located on the first body 2202. The reference markers 2702 can indicate superior-inferior distance between the base marker 2704 and the reference markers 2702 along the fourth axis A4. For example, the reference markers 2702 disposed on the first movable member 2206 can be scanned and compared to the base marker 2704 to determine the superior-inferior distance. In some implementations, the superior-inferior distance can indicate a distance between the base marker 2704 and the top surface 2220 or the cutting support indicator 2226 corresponding to “0.” For example, the superior-inferior distance can indicate a plane or level at which the cutting support member 2218 is guiding the cutting member 2502 to cut at. The superior-inferior distance can be used to determine the plane, and in some implementations, can be used to adjust the humeral head cutting apparatus 2200 to a desired plane for cutting the humeral head 110.

The reference markers 2702 can indicate an anterior-posterior distance between the base markers 2704 and reference markers 2702 that move along the fifth axis A5. For example, the reference markers 2702 disposed on the arm 2216 can be scanned and compared to the base marker 2704 to determine a distance between the reference marker 2702 and the base marker 2704 which can indicate the anterior-posterior distance. The anterior-posterior distance can indicate a lateral position of the cutting support member 2218, and the humeral head cutting apparatus 2200 can be adjusted based on the anterior-posterior distance to a desired lateral position. The base marker 2704 can also indicate a distance between the base marker 2704 and the fourth axis A4 as such distance may not change.

Referring further to FIG. 28, in some implementations, an angular position of the platform 2602 can be adjusted using the fourth fastener 2604. For example, in implementations where the cutting support member 2218 is adjusted to be angled via the third fastener 2234 or in implementations where the cutting member 2502 rests on and cuts the humeral head 110 while in contact with the platform 2602, an angular position of the platform 2602 can be adjusted to adjust an angle at which the humeral head 110 is cut. The third fastener 2234 can be released to allow for angular adjustment of the platform 2602 relative to the cutting support member 2218, and the third fastener 2234 can be tightened or otherwise actuated to lock the angular position of the platform 2602. In such implementations, the cutting support member 2218 can include a plurality of platform indicators 2802 to indicate the angular position of the platform 2602. For example, a user can adjust the platform 2602 such that a marker 2804 on the fourth fastener 2604 aligns with one of the platform indicators 2802. Alignment of the marker 2804 with at least one of the platform indicators 2802 can indicate an angular position of the platform 2602 relative to the cutting support member 2218.

In some implementations, the humeral head cutting apparatus 2200 can include a reference marker 2702 disposed at the fourth fastener 2604 or the platform 2602. The reference marker 2702 along with the base marker 2704 can indicate an angle of the platform 2602 relative to the first body 2202. In some implementations, the humeral head cutting apparatus 220 includes a reference marker 2702 disposed at the third fastener 2234. The reference marker 2702 along with the base marker 2704 can indicate an angle of the cutting support member 2218 relative to the first body 2202. In some implementations, the reference marker 2702 (e.g., first reference marker, etc.) can be coupled to and located on the cutting support member 2218 or the third fastener 2234. The reference marker 2702 can be scanned along with the base marker 2704 to determine an angle of the cutting support member 2218 relative to the first body 2202. The angle can correspond to an angle of the cutting plane relative to the humeral head 110 or the first body 2202. At least one of the reference markers 2702 are associated with an angle of the cutting support member 2218, such as being located on and coupled to at least one of the cutting support member 2218, the third fastener 2234, the platform 2602, or the fourth fastener 2604.

In implementations where the humeral head cutting apparatus 2200 includes the reference markers 2702, a surgeon can scan the reference markers 2702 and base markers 2704 using at least one sensor including at least one of a camera, VR device, AR device, mobile device, or any other computing system to determine the distances and an angle at which the humeral head cutting apparatus 2200 is set at relative to the humeral head 110. In some implementations, the computing system can be given a specific angle, such as by the surgeon, to cut the humeral head 110 at to harvest a core or graft of the humeral head 110 with specific dimensions, and the computing system can determine the angle and distances at which to set the humeral head cutting apparatus 2200 to create the desired graft. Consequently, the computing system or the surgeon can position the humeral head cutting apparatus 2200 at the output angle according to the desired graft.

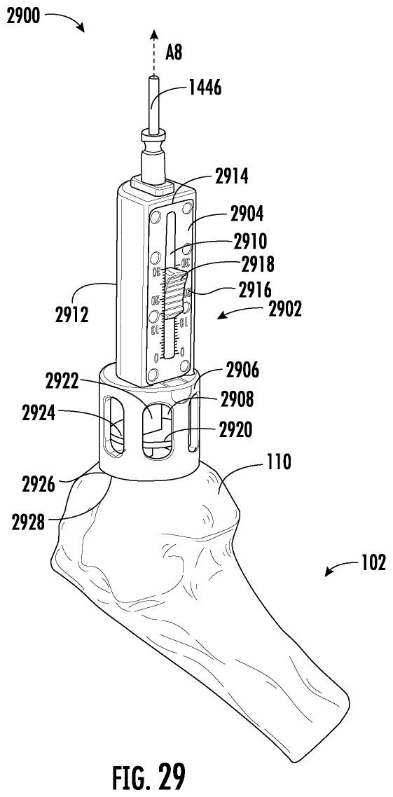

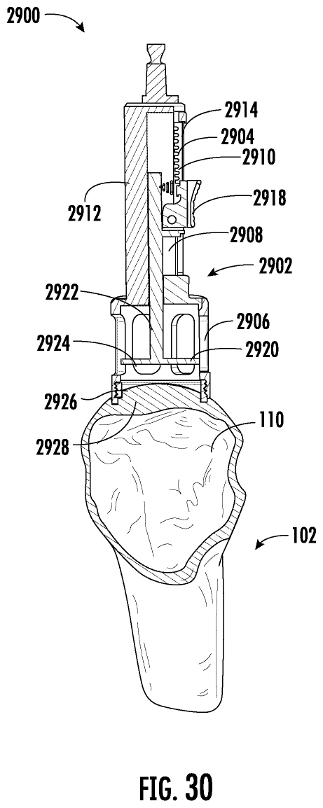

Referring now to FIGS. 29-30, FIGS. 29-30 depict various views of an example humeral head coring apparatus 2900 (e.g., coring drill, coring tool, etc.). The humeral head coring apparatus 2900 can be an example of coring drill 700. Prior to cutting of the humeral head by the humeral head cutting apparatus 2200, the humeral head surgical guide 1400 can be removed from the guide wire 1446, and the humeral head coring apparatus 2900 can be attached to the guide wire 1446. The humeral head coring apparatus 2900 can define a cut by coring at least a portion of at least one of the humeral head 110 or the humerus 102 to prepare the humerus 102 to provide a graft (e.g., bone graft, etc.).

The humeral head coring apparatus 2900 can include at least one body 2902. The body 2902 can be an example of main body 702. The body 2902 can extend along an eighth axis A8 which, in response to the humeral head coring apparatus 2900 being attached to the guide wire 1446, the guide wire 1446 can also extend along the eighth axis A8. The body 2902 can include one or more portions including a first portion 2904 and a second portion 2906 contiguous with the first portion 2904. A diameter of the second portion 2906 can be greater than the first portion 2904. In some implementations, the diameter of the second portion 2906 is less than or equal to the first portion 2904. A length of the first portion 2904 can be greater than a length of the second portion 2906. In some implementations, the length of the first portion 2904 is less than or equal to the length of the second portion 2906.

The body 2902 can define a body aperture 2908 which can extend along the eighth axis A8 (e.g., fourth axis). The body aperture 2908 can extend through the first portion 2904 and the second portion 2906 such that the body aperture 2908 receive the guide wire 1446. Consequently, the guide wire 1446 can extend through both the first portion 2904 and the second portion 2906, and out at least one of the first portion 2904 or the second portion 2906.

The first portion 2904 can define a track 2910 (e.g., guide rail, etc.). The track 2910 can extend along a length of the first portion 2904. In some implementations, the first portion 2904 includes a curved surface 2912 and a planar surface 2914. The track 2910 can be defined by the planar surface 2914. In some implementations, the first portion 2904 includes one or more indicators 2916 along the track 2910. The indicators 2916 can be an example of marks 704. For example, the one or more indicators 2916 can be oriented towards the track 2910 and be disposed along the length of the track 2910. In some implementations, the indicators 2916 are located on at least one of both or one side of the track 2910. The indicators 2916 can indicate a cutting depth. For example, the humeral head coring apparatus 2900 can be set to core out a portion of the humerus 102 or humeral head 110 (e.g., bone graft, etc.) with a height corresponding to the cutting depth.

The humeral head coring apparatus 2900 can include at least one adjuster 2918 coupled to and moving along the track 2910. The adjuster 2918 can be an example of adjusting button 706. The humeral head coring apparatus 2900 can include at least one receiving member 2920 (e.g., plunger) coupled to the adjuster 2918. The receiving member 2920 can be an example of plunger 708. The receiving member 2920 can extend at least partially through the first portion 2904 and the second portion 2906, and can include a rod 2922 and a plate 2924 coupled to the rod 2922. The plate 2924 can be contiguous with the rod 2922. The adjuster 2918 can be coupled with the rod 2922. For example, the adjuster 2918 can extend through the track 2910 to couple to the rod 2922.

The adjuster 2918 can move the receiving member 2920 and therefore set a cutting depth of the humeral head coring apparatus 2900. The adjuster 2918 can be at least one of a button, lever, or any other actuator, and in response to being actuated, such as a force being applied on the adjuster 2918 towards the rod 2922, the adjuster 2918 can allow movement of the receiving member 2920 along the track 2910. A position or height of the plate 2924 can determine the cutting depth, and the adjuster 2918 can position the receiving member 2920 according to the indicators 2916. The indicators 2916 can correspond to and indicate the cutting depth which corresponds to a position of the receiving member 2920. In response to being released, the adjuster 2918 can lock a position of the receiving member 2920. The receiving member 2920 can be adjusted by the adjuster 2918 by an operator (e.g., user, etc.), such as a surgeon or clinician, according to a desired cutting depth, such as a height of a bone graft.

The humeral head coring apparatus 2900 can include at least one coring member 2926 (e.g., cutting member, etc.). The coring member 2926 can be an example of insert 710. The coring member 2926 can include a plurality of cutting features 2928 (e.g., cutting edges, etc.), such as but not limited to teeth, knives, or any other features capable of cutting bone. The coring member 2926 can be coupled to the second portion 2906. For example, the coring member 2926 can extend around an entire periphery of the second portion 2906, and can be centered on the eighth axis A8. The coring member 2926 can core (e.g., cut, etc.) at least a portion of the humeral head 110 or the humerus 102 using the cutting features 2928. For example, the humeral head coring apparatus 2900 can be coupled to a drill, such as saw 2504. The first portion 2904 can be coupled to the drill, and in response to the drill providing current to the humeral head coring apparatus 2900, at least the coring member 2926 can rotate, allowing the coring member 2926 to cut into a portion of the humeral head 110.

In some implementations, the coring member 2926 is removable from the second portion 2906, such that the coring member 2926 can be replaced or adjusted. For example, cutting features 2928 can be replaced or otherwise adjusted, such as by changing a subset of the cutting features 2928 with a new or different type of cutting features 2928. In some implementations, a diameter of the coring member 2926 is adjustable, such as by swapping a coring member 2926 with a first diameter with a coring member 2926 of a second diameter. In some implementations, the diameter of the coring member 2926 and thus the diameter of the resected portion of the humerus 102 corresponds to a diameter of a component of the implant, such as the peg, keel, or baseplate.

While coring (e.g., cutting, etc.), the humeral head coring apparatus 2900 can move towards the humerus 102 while the cutting features 2928 rotate about the axis A8 until the humeral head 110 or the humerus 102 contacts the plate 2924. Upon contact with the plate 2924, the drill can stop providing power (e.g., as controlled by the user, etc.), and the humeral head coring apparatus 2900 can cut the portion of the humerus 102 or humeral head 110. For example, upon contact with the plate 2924, the operator can sense or feel a resistance, and determine that the cutting depth has been met as set by the adjuster 2918 and the receiving member 2920. The humeral head coring apparatus 2900 can then be removed from the guide wire 1446, and the cut created by the humeral head coring apparatus 2900 can be harvested as a bone graft following cutting. For example, following removal of the humeral head coring apparatus 2900, the humeral head cutting apparatus 2200 is attached, and the humeral head 110 can be cut using the humeral head cutting apparatus 2200. The humeral head 110 can be cut equal to or below a depth of the bone graft such that the bone graft can be harvested following resection of the humeral head 110.

In some implementations, the receiving member 2920 defines a receiving member aperture, and the guide wire 1446 extends through the receiving member aperture upon attachment of the humeral head coring apparatus 2900 with the guide wire 1446. In some implementations, the guide wire 1446 extends alongside the rod 2922, such as along an axis parallel to and offset from the rod 2922.

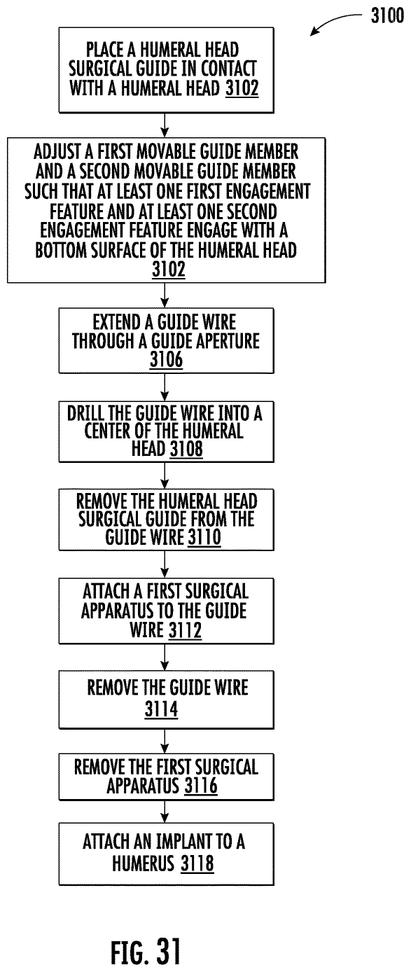

FIG. 31 depicts a flow diagram of an example method 3100 for preparing the humeral head 110 for an implant. In some implementations, the method 3100 can use a surgical kit including the humeral head surgical guide 1400, the humeral head cutting apparatus 2200 (e.g., first surgical apparatus), the humeral head coring apparatus 2900 (e.g., second surgical apparatus), a guide wire (e.g., guide wire 1446), and an implant (e.g., shoulder implant, humeral head implant, etc.). In some implementations, the method 3100 includes the method 2100.

The method 3100, at block 3102, can include placing a humeral head surgical guide in contact with a humeral head. For example, prior to the method 3100, skin and tissue surrounding and overlying the humeral head 110 may be moved or otherwise removed. The humeral head surgical guide can include a guide body including a first marker and a second marker, the guide body defining a guide aperture extending along a first axis at a center of the guide body, where the guide aperture receives the guide wire; a first movable guide member coupled to the guide body comprising one or more first indicators, where the first movable guide member moves along a second axis perpendicular to the first axis; a first supporting member including at least one first engagement feature, the first supporting member coupled to and extending from the first movable guide member, where the first marker is oriented relative to the one or more first indicators to indicate a first distance between the first axis and the at least one first engagement feature; a second movable guide member coupled to the guide body including one or more second indicators, where the second movable guide member moves along a third axis perpendicular to the first axis and relative to the second axis; and a second supporting member including at least one second engagement feature, the second supporting member coupled to and extending from the second movable guide member, where the second marker is oriented relative to the one or more second indicators to indicate a second distance between the first axis and the at least one second engagement feature.

The method 3100, at block 3104, can include adjusting a first movable guide member and a second movable guide member of the humeral head surgical guide such that at least one first engagement feature and at least one second engagement feature engage with a bottom surface of the humeral head. In some implementations, the method 3100 can include independently moving the first movable guide member and the second movable guide member. In some implementations, the method 3100 can include using a gear to maintain the at least one first engagement feature and the at least one second engagement feature at a same distance from the first axis and to move the first movable guide member and the second movable guide member at a same rate. In some implementations, the method 3100 can include engaging the at least one first engagement feature and the at least one second engagement feature with different portions of the bottom surface of the humeral head. In some implementations, the method 3100 can include using a first extender and a second extender, each coupled to the respective movable guide member, to reference anatomical locations on the humeral head.

In some implementations, the method 3100 can further include adjusting a third movable guide member and a fourth movable guide member of the humeral head surgical guide such that at least one third engagement feature the at least one fourth engagement feature engage with the bottom surface of the humeral head; and determining one or more dimensions of the humeral head based at least on the first distance, the second distance, the third distance, and the fourth distance. The method 3100 can further include selecting a shoulder implant (e.g., implant) based at least on the one or more dimensions.

The method 3100, at block 3106, can include extending a guide wire through a guide aperture of the humeral head surgical guide. In some implementations, the method 3100 can include receiving the guide wire in a shaft aperture of the humeral head surgical guide. The guide wire can be inserted through the humeral head surgical guide and into contact with a center of the humeral head as determined and set by the humeral head surgical guide.

The method 3100, at block 3108, can include drilling the guide wire into the center of the humeral head. In some implementations, the method 3100 can include determining a center of the humeral head based on adjustment of the first and second movable guide members, and drilling the guide wire into the determined center. In some implementations, the center can be determined based on adjustment of the first, second, third, and fourth movable guide members which can attach to the bottom surface of the humeral head via respective supporting members and engagement features, and be adjusted so as to determine the center.

The method 3100, at block 3110, can include removing the humeral head surgical guide from the guide wire. For example, the humeral head surgical guide can be removed from the guide wire following drilling of the guide wire into the humeral head and attachment of the guide wire at the center of the humeral head.

The method 3100, at block 3112, can include attaching a first surgical apparatus to the guide wire. For example, the first surgical apparatus can be inserted over the guide wire such that the guide wire extends through the first surgical apparatus. In some implementations, the method 3100 can include using the first surgical apparatus to cut the humeral head. In some implementations, the method 3100 can include adjusting the first surgical apparatus using at least a first movable member and a second movable member to set a position of a cutting support member to define a cutting plane. In some implementations, the method 3100 can include driving pins into a subset of cutting support apertures to secure the cutting support member to the humeral head.

The method 3100, at block 3114, can include removing the guide wire prior to cutting the humeral head. In some implementations, the method 3100 can include causing a cutting member to cut the humeral head along a top surface of the cutting support member. In some implementations, the method 3100 can include maintaining contact between the cutting member and the top surface while cutting the humeral head. In some implementations, the method 3100 can include adjusting the first surgical apparatus using at least one of a first fastener or a second fastener. The cutting of the first surgical apparatus can provide a planar surface of the humeral head for implant preparation. In some implementations, a portion of the first surgical apparatus is removed along with the guide wire prior to cutting, and the cutting support member can remain coupled to the humeral head via the pins.

The method 3100, at block 3116, can include removing the first surgical apparatus. The first surgical apparatus can be removed following cutting and removal of a portion of the humeral head such as to define a planar surface.

The method 3100, at block 3118, can include attaching an implant to a humerus. In some implementations, the method 3100 can include determining dimensions of the humeral head based at least on a first distance and a second distance, and selecting the implant based at least on the determined dimensions. In some implementations, the method 3100 can include determining dimensions of the humeral head based at least on the first distance, the second distance, and a third distance (e.g., thickness or depth of the humeral head 110, fifth distance).

In some implementations, such as implementations including harvesting a bone graft, the method 3100, can include attaching a second surgical apparatus to the guide wire prior to attaching the first surgical apparatus. For example, the second surgical apparatus can be inserted over the guide wire such that the guide wire extends through the second surgical apparatus. In some implementations, the method 3100 can include using the second surgical apparatus to cut (e.g., remove, core, etc.) at least a portion of the humeral head (e.g., bone graft).

In some implementations, the method 3100 can include moving an adjuster along the track to determine a position of a receiving member to define a cutting depth of the coring member. In some implementations, the method 3100 can include drilling, using the coring member, into the humerus around the guide wire and cutting, using the receiving member and the body, a portion of the humerus. In some implementations, the method 3100 can include removing the second surgical apparatus. The second surgical apparatus can be removed prior to attaching the first surgical apparatus.

For example, a surgeon can use the method 3100 to prepare the humeral head 110 and humerus 102 for implantation of a shoulder implant using at least one of the guide wire 1446, the humeral head surgical guide 1400, the humeral head coring apparatus 2900 (e.g., second surgical apparatus), or the humeral head cutting apparatus 2200 (e.g., first surgical apparatus). Following exposure of the humeral head 110 and removal of obstructing soft tissues and osteophytes, the surgeon can place and adjust the humeral head surgical guide 1400 to determine a center of the humeral head 110, measure one or more dimensions of the humeral head 110, and drill the guide wire 1446 into the center of the humeral head 110 as described above with respect to the humeral head surgical guide 1400 and method 2100. Once the guide wire 1446 has been drilled into the humeral head 110 and the humeral head surgical guide 1400 has been removed, the guide wire 1446 remains attached to the center of the humeral head 110 and serves as a reference axis for subsequent humeral preparation steps.

With the guide wire 1446 in place, the surgeon can optionally attach the second surgical apparatus, such as the humeral head coring apparatus 2900, to the guide wire 1446 prior to resection of the humeral head 110. The body aperture 2908 of the body 2902 can be advanced over the guide wire 1446 so that the first portion 2904 and the second portion 2906 of the body 2902 are coaxially aligned with the guide wire 1446 along the axis A8. The coring member 2926, coupled to and extending around the second portion 2906, is thereby positioned adjacent to the humeral head 110 and/or humerus 102. In this configuration, the guide wire 1446 constrains the humeral head coring apparatus 2900 so that the coring member 2926 cores bone in a path centered on the previously established center of the humeral head 110.

Once the humeral head coring apparatus 2900 is positioned on the guide wire 1446, the surgeon can set the desired cutting depth of the coring member 2926 by adjusting the receiving member 2920 using the adjuster 2918 along the track 2910 of the first portion 2904. In some implementations, the surgeon can press or otherwise actuate the adjuster 2918 to release the adjuster 2918 from a locked position and slide the adjuster 2918 along the track 2910 until a desired indicator 2916 corresponding to a target cutting depth is reached. Alignment of the adjuster 2918 with a selected indicator 2916 can correspond to a particular position of the receiving member 2920 (e.g., rod 2922 and plate 2924) within the body 2902, thereby defining the height of bone that will be cored. The adjuster 2918 can then be released to lock the receiving member 2920 in the chosen position so that the plate 2924 acts as a mechanical stop for the coring member 2926 at the predetermined cutting depth.

With the cutting depth set, the surgeon can couple the humeral head coring apparatus 2900 to a drill via an attachment on the first portion 2904 and activate the drill so that at least the coring member 2926 and cutting features 2928 rotate about the axis A8 around the guide wire 1446. The surgeon can then advance the body 2902 toward the humeral head 110 such that the coring member 2926 cores into the humeral head 110 and/or humerus 102 concentric with the guide wire 1446. As the coring member 2926 advances, the cutting features 2928 can excise a cylindrical or otherwise shaped portion of bone with a diameter corresponding to the outer diameter of the coring member 2926, while the guide wire 1446 maintains alignment with the previously established central axis of the humeral head 110. Advancement can continue until the cored bone portion contacts the plate 2924 of the receiving member 2920, indicating that the preset cutting depth has been reached.

Once the desired depth has been achieved, the surgeon can stop the drill and withdraw the humeral head coring apparatus 2900 along the guide wire 1446 away from the humeral head 110. In some implementations, the cored bone portion can be preserved as a bone graft for subsequent use, for example in glenoid reconstruction.

After coring is complete, the surgeon can remove the humeral head coring apparatus 2900 from the guide wire 1446, leaving the guide wire 1446 in place at the center of the humeral head 110. The guide wire 1446 can continue to serve as a reference axis for subsequent resection. In some implementations, the humeral head coring apparatus 2900 and the cored bone portion are removed together, and the surgeon can inspect or measure the graft outside the patient.

With the coring phase complete (if used), the surgeon can then attach the first surgical apparatus, such as the humeral head cutting apparatus 2200, to the guide wire 1446 for controlled resection of the humeral head 110. The first body 2202 of the humeral head cutting apparatus 2200 can be advanced over the guide wire 1446 so that the guide wire 1446 extends through the first aperture 2204 and the first body 2202 is aligned along the axis A4 with the guide wire 1446. The first movable member 2206, second body 2208, second movable member 2210, arm 2216, and cutting support member 2218 can thereby be positioned relative to the humeral head 110 with reference to the central axis established by the guide wire 1446.

Once the humeral head cutting apparatus 2200 is mounted on the guide wire 1446, the surgeon can adjust the position and orientation of the cutting support member 2218 to define a desired cutting plane for humeral head resection. For example, the surgeon can loosen the first fastener 2212 and slide the first movable member 2206 along the first body 2202 until a body indicator 2228 corresponding to a desired resection level (e.g., related to the measured depth of the humeral head 110 obtained from fifth indicators 1450) is visible in the movable member aperture 2230 and aligned with the marker 2232. The first fastener 2212 can then be tightened to lock the vertical position of the cutting support member 2218. Similarly, the surgeon can loosen the second fastener 2214 and slide the second movable member 2210 along the second body 2208 to adjust the lateral or anteroposterior location of the arm 2216 and cutting support member 2218, then tighten the second fastener 2214 to fix that position.

The surgeon can further set the inclination or version angle of the cutting plane by loosening the third fastener 2234 and rotating the cutting support member 2218 relative to the arm 2216 until a desired angular orientation is reached. In some implementations, the angular position of a platform 2602, when included in the humeral head cutting apparatus 2200, can also be adjusted using the fourth fastener 2604 and platform indicators 2802 to further refine the cutting plane. Once the desired angular orientation is achieved, the third fastener 2234 (and fourth fastener 2604 in some implementations) can be tightened to lock the cutting support member 2218 and platform 2602 in place. The combined settings of first movable member 2206, second movable member 2210, and third fastener 2234, and optional fourth fastener 260, can therefore define a cutting plane for top surface 2220, and in some implementations, platform 2602 that corresponds to a planned humeral head resection level, orientation, and offset.

After the cutting support member 2218 is positioned and oriented, the surgeon can select one or more cutting support apertures 2222 based on bone quality, desired offset, and cutting indicators 2226, and can use pins 2236 to secure the cutting support member 2218 to the humeral head 110. For example, pins 2236 can be driven through a subset of the cutting support apertures 2222 (e.g., those associated with a cutting indicator 2226 of “0” or an offset of +2 or −2 mm) and into the humeral head 110, thereby fixing the second surface 2402 of the cutting support member 2218 against the bone. Once the cutting support member 2218 is secured with pins 2236, the first body 2202, the first movable member 2206, the second body 2208, and the second movable member 2210 can be disconnected from the cutting support member 2218 by loosening and removing the third fastener 2234, and, in some implementations, the fourth fastener 2604, and sliding the proximal portion of the humeral head cutting apparatus 2200 off the guide wire 1446.

With the cutting support member 2218 pinned to the humeral head 110 and the proximal portion of the humeral head cutting apparatus 2200 removed, the surgeon can remove the guide wire 1446 from the humeral head 110. The surgeon can then position a cutting member 2502, such as an oscillating saw blade, so that the cutting member 2502 rests on and is guided by at least the top surface 2220 and, in some implementations, the platform 2602. The cutting member 2502 can be actuated and advanced along the top surface 2220 such that the humeral head 110 is resected along the defined cutting plane. In some implementations, the cutting member 2502 can maintain contact with the top surface 2220 throughout cutting, thereby ensuring that the resection follows the intended plane and depth.

Once the humeral head 110 has been resected along the cutting plane defined by the cutting support member 2218, the surgeon can stop the cutting member 2502 and withdraw the cutting member 2502 from the humeral head 110 and contact with the cutting support member 2218. The pins 2236 can then be removed from the humeral head 110, and the cutting support member 2218 can be detached and removed, leaving a planar surface on the resected humerus 102. In implementations including the humeral head coring apparatus 2900 previously used to core a portion of the humeral head 110 and/or humerus 102, the resection plane can intersect the previously cored region so that the cored bone portion can be fully separated and extracted as a bone graft.

After completion of the coring and cutting steps, the surgeon can use the dimensional measurements obtained with the humeral head surgical guide 1400 (e.g., first, second, third, fourth, and fifth distances) together with the prepared planar surface to select an implant of appropriate size and geometry. The implant can then be positioned on or within the humerus 102, for example with a humeral head or articular component aligned with the planar resection surface. The implant can be attached using known fixation techniques (e.g., press-fit, cemented).

In some implementations, the methods 2100 and 3100 can be perfumed by a surgeon, in an augmented reality (AR) or virtual reality (VR) environment, or by a surgical robot. For example, in situations where the methods 2100 or 3100 are performed an augmented reality (AR) or virtual reality (VR) environment, or by a surgical robot, one or more sensors can scan the reference markers 2002 to determine placement of the guide wire 1446, cutting of the humeral head 110, etc. In some implementations, the guide wire 1446 can be attached to the humeral head 110 prior to attachment of the humeral head 110. For example, one or more imaging systems, imaging scans, or other guidance systems can determine the center of the humeral head 110, and the guide wire 1446 can be drilled into the determined center of the humeral head 110.

In some implementations, one or more machine learning models, such as prediction models, can be used to determine the center of the humeral head 110. The models can be updated using images of the humeral head 110 and corresponding dimensions measured using the humeral head surgical guide 1400. For example, the models can receive the images of the humeral head 110, and output dimensions that also indicate the center of the humeral head surgical guide 1400. Consequently, the center of the humeral head 110 can be determined and guide wire 1446 can be drilled into the center of the humeral head 110 without use of the humeral head surgical guide 1400. The models can be update continuously updated with images of the humeral head 110 taken by one or more sensors following exposure of the humeral head 110 to improve accuracy.

At least one aspect relates to a method. The method can include placing a humeral head surgical guide in contact with a humeral head. The humeral head surgical guide can include a guide body including a first marker and a second marker. The guide body can define a guide aperture extending along a first axis at a center of the guide body. The humeral head surgical guide can include a first movable guide member coupled to the guide body including one or more first indicators. The first movable guide member can move along a second axis perpendicular to the first axis. The humeral head surgical guide can include a first supporting member including at least one first engagement feature. The first supporting member can be coupled to and extend from the first movable guide member. The first marker can be oriented relative to the one or more first indicators to indicate a first distance between the first axis and the at least one first engagement feature. The humeral head surgical guide can include a second movable guide member coupled to the guide body including one or more second indicators. The second movable guide member can move along a third axis perpendicular to the first axis and relative to the second axis. The humeral head surgical guide can include a second supporting member including at least one second engagement feature. The second supporting member can be coupled to and extend from the second movable guide member. The second marker can be oriented relative to the one or more second indicators to indicate a second distance between the first axis and the at least one second engagement feature. The method can include adjusting the first movable guide member and the second movable guide member such that the at least one first engagement feature and the at least one second engagement feature engage with a bottom surface of the humeral head. The method can include extending a guide wire through the guide aperture. The method can include drilling the guide wire into the humeral head.

In some implementations, the method can include adjusting a shaft of the humeral head surgical guide including one or more fifth indicators such that the shaft contacts a top surface of the humeral head. In some implementations, the method can include determining a depth of the humeral head based at least on the one or more fifth indicators indicating a fifth distance between the shaft and at least one of the at least one first engagement feature and the at least one second engagement feature.

In some implementations, the guide body further includes a third marker and a fourth marker. In some implementations, the humeral head surgical guide can include a third movable guide member coupled to the guide body including one or more third indicators. In some implementations, the third movable guide member can move along the first axis. In some implementations, the humeral head surgical guide can include a third supporting member including at least one third engagement feature. In some implementations, the third supporting member can be coupled to and extend from the third movable guide member. In some implementations, the third marker can be oriented relative to the one or more third indicators to indicate a third distance between the first axis and the at least one third engagement feature. In some implementations, the humeral head surgical guide can include a fourth movable guide member coupled to the guide body including one or more fourth indicators. In some implementations, the fourth movable guide member can move along the second axis. In some implementations, the humeral head surgical guide can include a fourth supporting member including at least one fourth engagement feature. In some implementations, the fourth supporting member can be coupled to and extend from the fourth movable guide member. In some implementations, the fourth marker can be oriented relative to the one or more fourth indicators to indicate a fourth distance between the first axis and the at least one fourth engagement feature.

In some implementations, the method can include adjusting the third movable guide member and the fourth movable guide member such that the at least one third engagement feature and the at least one fourth engagement feature engage with the bottom surface of the humeral head. In some implementations, the method can include determining one or more dimensions of the humeral head based at least on the first distance, the second distance, the third distance, and the fourth distance.

In some implementations, the method can include selecting a shoulder implant based at least on the one or more dimensions. In some implementations, the method can include implanting the shoulder implant.

In some implementations, the humeral head surgical guide further includes a gear coupled with the first movable guide member and the second movable guide member. In some implementations, the gear can maintain the at least one first engagement feature and the at least one second engagement feature at a same distance from the first axis. In some implementations, the gear can cause the first movable guide member and the second movable guide member to be movable at a same rate.

In some implementations, adjustment of at least the first movable guide member and the second movable guide member determines a center of the humeral head. In some implementations, the guide wire is drilled into the center of the humeral head.

In some implementations, the humeral head surgical guide further includes a base marker coupled to and located on the guide body, a first reference marker coupled to and located on the first extender, a second reference marker coupled to and located on the second extender, a shaft coupled to the guide body, a third handle coupled to and extending around the shaft, and a third reference marker coupled to and located on the third handle where to determine at least the first distance, a fourth distance, and a fifth distance, the method further includes scanning, using at least one sensor, the base marker and the first reference marker to determine the first distance, scanning, using the at least one sensor, the base marker and the second reference marker to determine the fourth distance, and scanning, using the at least one sensor, the base marker and the third reference marker to determine at least the fifth distance, where the first distance, the fourth distance, and the fifth distance are used to at least select an implant.

At least one aspect relates to a method. The method can include placing a humeral head surgical guide in contact with a humeral head to determine a center of the humeral head. The humeral head surgical guide can include a guide body including a first marker and a second marker. The guide body can define a guide aperture extending along a first axis at a center of the guide body. The humeral head surgical guide can include a first movable guide member coupled to the guide body including one or more first indicators. The first movable guide member can move along a second axis perpendicular to the first axis. The humeral head surgical guide can include a first supporting member including at least one first engagement feature. The first supporting member can be coupled to and extend from the first movable guide member. The first marker can be oriented relative to the one or more first indicators to indicate a first distance between the first axis and the at least one first engagement feature. The humeral head surgical guide can include a second movable guide member coupled to the guide body including one or more second indicators. The second movable guide member can move along a third axis perpendicular to the first axis and relative to the second axis. The humeral head surgical guide can include a second supporting member including at least one second engagement feature. The second supporting member can be coupled to and extend from the second movable guide member. The second marker can be oriented relative to the one or more second indicators to indicate a second distance between the first axis and the at least one second engagement feature. The method can include adjusting the first movable guide member and the second movable guide member such that the at least one first engagement feature and the at least one second engagement feature engage with a bottom surface of the humeral head. The method can include extending a guide wire through the guide aperture. The method can include drilling the guide wire into a center of the humeral head. The method can include removing the humeral head surgical guide from the guide wire. The method can include attaching a first surgical apparatus to the guide wire. The first surgical apparatus can cut the humeral head. The method can include removing the guide wire prior to cutting the humeral head. The method can include attaching an implant to a humerus.

In some implementations, the first surgical apparatus can include a first body defining a first aperture. In some implementations, the first surgical apparatus can include a first movable member extending around the first body. In some implementations, the first movable member can be movable along the first body. In some implementations, the first surgical apparatus can include a second body coupled to the first movable member. In some implementations, the first surgical apparatus can include a second movable member extending around the second body. In some implementations, the second movable member can be movable along the second body. In some implementations, the first surgical apparatus can include an arm coupled to the second movable member. In some implementations, the first surgical apparatus can include a cutting support member removably coupled with the arm. In some implementations, the cutting support member can define a plurality of cutting support apertures. In some implementations, the cutting support member can include a top surface that guides a cutting member. In some implementations, the cutting support member can include a plurality of pins extending through a subset of the plurality of cutting support apertures. In some implementations, the plurality of pins can secure the cutting support member to a humeral head.

In some implementations, to cut the humeral head, the method can further include adjusting the first surgical apparatus using at least the first movable member and the second movable member to set a position of the cutting support member to define a cutting plane of the cutting member, driving the plurality of pins into the subset of the plurality of cutting support apertures to secure the cutting support member to the humeral head, removing a portion of the first surgical apparatus and the guide wire, and causing the cutting member to cut the humeral head along the top surface.

In some implementations, the first surgical apparatus further includes a base marker coupled to and located on the first body and a reference marker associated with an angle of the cutting support member relative to the first body, the method further including scanning, using at least one sensor, the base marker and the reference marker to determine the angle.

In some implementations, the cutting member is in contact with the top surface while cutting the humeral head. In some implementations, the first surgical apparatus can further include a first fastener coupled to the first movable member and a second fastener coupled to the second movable member, the method further including adjusting the first surgical apparatus using at least one of the first fastener or the second fastener.

In some implementations, the method further includes prior to attaching the first surgical apparatus, attaching a second surgical apparatus to the guide wire, where the second surgical apparatus cores the humeral head, in response to coring the humeral head, removing the second surgical apparatus, and in response to cutting the humeral head, extracting a bone graft of the humeral head corresponding to the coring of the humeral head.

In some implementations, the second surgical apparatus can include a body including a first portion and a second portion contiguous with the first portion. In some implementations, the first portion can define a track. In some implementations, the body can define a body aperture extending along a first axis through the first portion and the second portion. In some implementations, the body aperture can receive a guide wire attached to a center of a humeral head. In some implementations, the second surgical apparatus can include a coring member coupled to the second portion. In some implementations, the coring member can comprise a plurality of cutting features. In some implementations, the coring member can core at least a portion of the humeral head using the plurality of cutting features. In some implementations, the second surgical apparatus can include an adjuster coupled to and movable along the track. In some implementations, the second surgical apparatus can include a receiving member coupled to the adjuster. In some implementations, the receiving member can define a cutting depth of the coring member. In some implementations, the receiving member can be movable by the adjuster to define the cutting depth.

In some implementations, the method further includes drilling, using the coring member, into the humerus around the guide wire and coring, using the receiving member and the body, a portion of the humerus.

In some implementations, the method can include determining dimensions of the humeral head based at least on the first distance and the second distance. In some implementations, the method can include selecting the implant based at least on the dimensions of the humeral head.

While this specification contains many specific implementation details, these should not be construed as limitations on the scope of what can be claimed but rather as descriptions of features specific to particular implementations. Certain features described in this specification in the context of separate implementations can also be implemented in combination in a single implementation. Conversely, various features described in the context of a single implementation can also be implemented in multiple implementations separately or in any suitable subcombination. Moreover, although features can be described as acting in certain combinations and even initially claimed as such, one or more features from a claimed combination can, in some cases, be excised from the combination, and the claimed combination can be directed to a subcombination or variation of a subcombination.

As utilized herein, the terms “substantially,” “generally,” “approximately,” “about,” and similar terms are intended to have a broad meaning in harmony with the common and accepted usage by those of ordinary skill in the art to which the subject matter of this disclosure pertains. It should be understood by those of skill in the art who review this disclosure that these terms are intended to allow a description of certain features described and claimed without restricting the scope of these features to the precise numerical ranges provided. Accordingly, these terms should be interpreted as indicating that insubstantial or inconsequential modifications or alterations of the subject matter described and claimed are considered to be within the scope of the appended claims.

The term “coupled” and the like, as used herein, mean the joining of two components directly or indirectly to one another. Such joining can be stationary (e.g., permanent) or moveable (e.g., removable or releasable). Such joining can be achieved with the two components or the two components and any additional intermediate components being integrally formed as a single unitary body with one another, with the two components, or with the two components and any additional intermediate components being attached to one another.

It is important to note that the construction and arrangement of the various systems shown in the various example implementations is illustrative only and not restrictive in character. All changes and modifications that come within the spirit and/or scope of the described implementations are desired to be protected. It should be understood that some features may not be necessary, and implementations lacking the various features can be contemplated as within the scope of the disclosure, the scope being defined by the claims that follow. When the language “a portion” is used, the item can include a portion and/or the entire item unless specifically stated to the contrary.

Also, the term “or” is used, in the context of a list of elements, in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. can be either X, Y, Z, X and Y, X and Z, Y and Z, or X, Y, and Z (i.e., any combination of X, Y, and Z). Thus, such conjunctive language is not generally intended to imply that certain implementations require at least one of X, at least one of Y, and at least one of Z to each be present, unless otherwise indicated.

Additionally, the use of ranges of values (e.g., W1 to W2, etc.) herein are inclusive of their maximum values and minimum values (e.g., W1 to W2 includes W1 and includes W2, etc.), unless otherwise indicated. Furthermore, a range of values (e.g., W1 to W2, etc.) does not necessarily require the inclusion of intermediate values within the range of values (e.g., W1 to W2 can include only W1 and W2, etc.), unless otherwise indicated.

1. A humeral head surgical guide, comprising:
a guide body comprising a first marker and a second marker, the guide body defining a guide aperture extending along a first axis at a center of the guide body;
a first movable guide member coupled to the guide body comprising one or more first indicators, wherein the first movable guide member moves along a second axis perpendicular to the first axis;
a first supporting member comprising at least one first engagement feature, the first supporting member coupled to and extending from the first movable guide member, wherein the first marker is oriented relative to the one or more first indicators to indicate a first distance between the first axis and the at least one first engagement feature;
a second movable guide member coupled to the guide body comprising one or more second indicators, wherein the second movable guide member moves along a third axis perpendicular to the first axis and relative to the second axis; and
a second supporting member comprising at least one second engagement feature, the second supporting member coupled to and extending from the second movable guide member, wherein the second marker is oriented relative to the one or more second indicators to indicate a second distance between the first axis and the at least one second engagement feature.

2. The humeral head surgical guide of claim 1, wherein the guide body comprises a third marker, the humeral head surgical guide further comprising:
a third movable guide member coupled to the guide body comprising one or more third indicators, wherein the third movable guide member moves along the second axis; and
a third supporting member comprising at least one third engagement feature, the third supporting member coupled to and extending from the third movable guide member, wherein the third marker is oriented relative to the one or more third indicators to indicate a third distance between the first axis and the at least one third engagement feature.

3. The humeral head surgical guide of claim 2, wherein the guide body comprises a fourth marker, the humeral head surgical guide further comprising:
a fourth movable guide member coupled to the guide body comprising one or more fourth indicators, wherein the fourth movable guide member moves along the third axis; and
a fourth supporting member comprising at least one fourth engagement feature, the fourth supporting member coupled to and extending from the fourth movable guide member, wherein the fourth marker is oriented relative to the one or more fourth indicators to indicate a fourth distance between the first axis and the at least one fourth engagement feature.

4. The humeral head surgical guide of claim 1, wherein the guide body is configured to receive a guide wire extending through the guide aperture, the guide wire attachable to the center of a humeral head using at least the first movable guide member and the second movable guide member.

5. The humeral head surgical guide of claim 4, further comprising:
a shaft defining a shaft aperture, the guide wire extending through the shaft aperture, the shaft comprising one or more fifth indicators, a first end, and a second end opposite the first end, wherein the one or more fifth indicators indicate a fifth distance between the second end and at least one of the at least one first engagement feature or the at least one second engagement feature.

6. The humeral head surgical guide of claim 5, wherein the fifth distance indicates a depth of the humeral head.

7. The humeral head surgical guide of claim 1, wherein the first movable guide member and the second movable guide member are independently movable.

8. The humeral head surgical guide of claim 1, further comprising:
a gear coupled with the first movable guide member and the second movable guide member, wherein the gear maintains the at least one first engagement feature and the at least one second engagement feature at a same distance from the first axis, wherein the gear causes the first movable guide member and the second movable guide member to move at a same rate.

9. The humeral head surgical guide of claim 1, wherein the at least one first engagement feature and the at least one second engagement feature engage with different portions of a bottom surface of a humeral head.

10. The humeral head surgical guide of claim 1, further comprising:
a first extender coupled to and extending from the first movable guide member, the first extender indicative of a first anatomical location on a humeral head; and
a second extender coupled to and extending from the second movable guide member, the second extender indicative of a second anatomical location on the humeral head.

11. The humeral head surgical guide of claim 10, further comprising:
a base marker coupled to and located on the guide body;
a first reference marker coupled to and located on the first extender, wherein the base marker and the first reference marker are scanned using at least one sensor to determine at least the first distance;
a second reference marker coupled to and located on the second extender, wherein the base marker and the second reference marker are scanned using the at least one sensor to determine at least a fourth distance;
a shaft coupled to the guide body;
a third handle coupled to and extending around the shaft; and
a third reference marker coupled to and located on the third handle, wherein the base marker and the third reference marker are scanned using the at least one sensor to determine at least a fifth distance.

12. A humeral head cutting apparatus, comprising:
a first body defining a first aperture;
a first movable member extending around the first body, the first movable member movable along the first body;
a second body coupled to the first movable member;
a second movable member extending around the second body, the second movable member movable along the second body;
an arm coupled to the second movable member; and
a cutting support member removably coupled with the arm, wherein the cutting support member defines a plurality of cutting support apertures, the cutting support member comprising:
a top surface that guides a cutting member; and
a plurality of pins extending through a subset of the plurality of cutting support apertures, wherein the plurality of pins secure the cutting support member to a humeral head.

13. The humeral head cutting apparatus of claim 12, wherein the plurality of cutting support apertures are defined at different positions on the cutting support member.

14. The humeral head cutting apparatus of claim 12, further comprising:
a first fastener coupled to the first movable member;
a second fastener coupled to the second movable member; and
a third fastener coupled to the cutting support member.

15. The humeral head cutting apparatus of claim 12, further comprising:
a platform removably coupled with the cutting support member and facing the top surface, wherein the cutting member extends between the top surface and the platform; and
a fourth fastener coupled to the platform, the fourth fastener removably coupling the platform from the cutting support member.

16. The humeral head cutting apparatus of claim 15, wherein the first aperture receives a guide wire attached to a center of the humeral head.

17. The humeral head cutting apparatus of claim 15, wherein the first body includes one or more first indicators indicating a depth, wherein a position of the first movable member along the first body is set to one of the one or more first indicators corresponding to a depth of the humeral head.

18. The humeral head cutting apparatus of claim 12, further comprising:
a base marker coupled to and located on the first body; and
a reference marker associated with an angle of the cutting support member relative to the first body, wherein the base marker and the reference marker are scanned to determine the angle.

19. A humeral head coring apparatus, comprising:
a body comprising a first portion and a second portion contiguous with the first portion, the first portion defining a track, the body defining a body aperture extending along a first axis through the first portion and the second portion, wherein the body aperture receives a guide wire attached to a center of a humeral head;
a coring member coupled to the second portion, the coring member comprising a plurality of cutting features, wherein the coring member cores at least a portion of the humeral head using the plurality of cutting features;
an adjuster coupled to and movable along the track; and
a receiving member coupled to the adjuster, the receiving member defining a cutting depth of the coring member, the receiving member movable by the adjuster to define the cutting depth.

20. The humeral head coring apparatus of claim 19, wherein the first portion comprises one or more indicators along the track, wherein the one or more indicators correspond to the cutting depth.

21. The humeral head coring apparatus of claim 19, wherein the second portion receives the portion of the humeral head, the portion having a height corresponding to the cutting depth, wherein the receiving member contacts the portion.

22. The humeral head coring apparatus of claim 19, wherein a diameter of the second portion is greater than a diameter of the first portion.

23. A surgical kit, comprising:
a guide wire;
a humeral head surgical guide, comprising:
a guide body comprising a first marker and a second marker, the guide body defining a guide aperture extending along a first axis at a center of the guide body, wherein the guide aperture receives the guide wire;
a first movable guide member coupled to the guide body comprising one or more first indicators, wherein the first movable guide member moves along a second axis perpendicular to the first axis;
a first supporting member comprising at least one first engagement feature, the first supporting member coupled to and extending from the first movable guide member, wherein the first marker is oriented relative to the one or more first indicators to indicate a first distance between the first axis and the at least one first engagement feature;
a second movable guide member coupled to the guide body comprising one or more second indicators, wherein the second movable guide member moves along a third axis perpendicular to the first axis and relative to the second axis; and
a second supporting member comprising at least one second engagement feature, the second supporting member coupled to and extending from the second movable guide member, wherein the second marker is oriented relative to the one or more second indicators to indicate a second distance between the first axis and the at least one second engagement feature; and
a first surgical apparatus, comprising:
a first body defining a first aperture, wherein the guide wire extends through the first aperture;
a first movable member extending around the first body, the first movable member movable along the first body; and
a cutting support member removably coupled with the first movable member, wherein the cutting support member guides a cutting member in cutting a humeral head;
a second surgical apparatus, comprising:
a body comprising a first portion and a second portion contiguous with the first portion, the first portion defining a track, the body defining a body aperture extending along a fourth axis through the first portion and the second portion, wherein the body aperture receives the guide wire attached to the center of the humeral head; and
a coring member coupled to the second portion, the coring member comprising a plurality of cutting features, wherein the coring member removes at least a portion of the humeral head using the plurality of cutting features.

24. The surgical kit of claim 23, the first surgical apparatus further comprising:
a second body coupled to the first movable member;
a second movable member extending around the second body, the second movable member movable along the second body; and
an arm coupled to the second movable member,
wherein the cutting support member is removably coupled to the arm.

25. The surgical kit of claim 23, wherein the cutting support member defines a plurality of cutting support apertures, the cutting support member comprising:
a top surface that guides the cutting member; and
a plurality of pins extending through a subset of the plurality of cutting support apertures, wherein the plurality of pins secure the cutting support member to the humeral head.

26. The surgical kit of claim 23, the second surgical apparatus further comprising:
an adjuster coupled to the track, the adjuster movable along the track; and
a receiving member coupled to the adjuster, the receiving member defining a cutting depth of the coring member, the receiving member movable by the adjuster to define the cutting depth.

27. The surgical kit of claim 23, wherein the first distance and the second distance indicate one or more dimensions of the humeral head, the surgical kit further comprising an implant, wherein the implant has dimensions corresponding to the one or more dimensions.

28. The surgical kit of claim 23, wherein the humeral head surgical guide further comprises:
a first extender coupled to and extending from the first movable guide member, the first extender indicative of a first anatomical location on a humeral head; and
a second extender coupled to and extending from the second movable guide member, the second extender indicative of a second anatomical location on the humeral head.

29. The surgical kit of claim 28, wherein the humeral head surgical guide further comprises:
a base marker coupled to and located on the guide body;
a first reference marker coupled to and located on the first extender, wherein the base marker and the first reference marker are scanned using at least one sensor to determine at least the first distance;
a second reference marker coupled to and located on the second extender, wherein the base marker and the second reference marker are scanned using the at least one sensor to determine at least a fourth distance;
a shaft coupled to the guide body;
a third handle coupled to and extending around the shaft; and
a third reference marker coupled to and located on the third handle, wherein the base marker and the third reference marker are scanned using the at least one sensor to determine at least a fifth distance.

30. The surgical kit of claim 23, wherein the first surgical apparatus further comprises:
a base marker coupled to and located on the first body; and
a reference marker associated with an angle of the cutting support member relative to the first body, wherein the base marker and the reference marker are scanned to determine the angle.
